(19) United States
(12) Patent Application Publication
(10) Pub. No.: US 2026/0253485 A1
(43) Pub. Date: Aug. 27, 2026

(54) METHOD AND SYSTEM FOR EMERGENCY RESPONSE MANAGEMENT

(71) Applicant: Alegion Health Tech Private Limited, Chennai (IN)

(72) Inventors: Raveendran NARASIMHAN, Chennai (IN); Saumyadeep DATTA, Chennai (IN)

(73) Assignee: Alegion Health Tech Private Limited, Chennai, TN (IN)

(21) Appl. No.: 19/298,243

(22) Filed: Aug. 13, 2025

Related U.S. Application Data
(63) Continuation-in-part of application No. PCT/IB2025/054177, filed on Apr. 22, 2025.

(30) Foreign Application Priority Data
Feb. 21, 2025 (IN) 202543015234

(51) Int. Cl. G08B 25/01; G16H 10/60; G16H 20/40; G16H 40/20; G16H 50/30

(52) U.S. Cl. CPC G08B 25/016; G16H 10/60; G16H 20/40; G16H 40/20; G16H 50/30

(57) ABSTRACT

Aspects of the present disclosure provide techniques for managing emergency response services. In an embodiment, emergency information related to a patient is received, where the emergency information contains one or more of: description of injuries of the patient, description of health issues of the patient, and visual media depicting present state of the patient. A location of the patient is determined. Health condition of the patient is assessed based at least in part on the received emergency information. A category of hospitals and a category of ambulances are selected based at least in part on the assessed health condition of the patient. A first alert is sent to one or more ambulances of the selected category of ambulances, the first alert including the location of the patient, and one or more hospitals from the selected category of hospitals.

### PRIORITY STATEMENT

The present application claims priority under 35 U.S.C. § 119 to PCT application No. PCT/IB2025/054177 filed on 22 Apr. 2025, that claims priority from Indian patent application number 202543015234 filed on 21 Feb. 2025 the entire contents of which are hereby incorporated herein by reference.

### FIELD

The present disclosure relates to emergency response services. More particularly the present invention relates to a system and method for emergency response management.

### BACKGROUND

In the realm of current technologies, emergency response systems typically focus on distinct aspects of emergency response management (including emergency response services management), such as notifying emergency contacts, dispatching ambulances, or directing to nearby medical facilities/hospitals etc. In the existing systems, there is a notable absence of integration of various emergency response services (such as emergency notifications to family and friends, emergency medical service dispatch, etc.) into a seamless operational flow.

Also, existing emergency response systems do not assess the severity of a patient's condition in real-time, and do not ensure that the ambulance dispatched is adequately equipped for the patient's specific needs. This limitation can lead to significant delays in providing necessary medical attention, potentially exacerbating the patient's condition or resulting in preventable fatality.

Further, existing emergency response systems do not determine appropriate medical facilities/hospitals based on the real-time medical needs of the patient. This deficiency underscores a significant gap in the ability to provide timely and integrated emergency care.

Therefore, there is a need for improved techniques for emergency response management. Aspects of the present disclosure are related to improved techniques for emergency response management.

### SUMMARY

The following summary is illustrative only and is not intended to be in any way limiting. In addition to the illustrative aspects, example embodiments, and features described, further aspects, example embodiments, and features, will become apparent by reference to the drawings and the following detailed description.

An aspect of the present disclosure provides for a method for managing emergency response services, the method performed at a system. In an embodiment, the method includes receiving emergency information related to a patient, where the emergency information contains one or more of: description of injuries of the patient, description of health issues of the patient, and visual media depicting present state of the patient. The method also includes determining a location of the patient, assessing health condition of the patient based at least in part on the received emergency information, and selecting a category of hospitals and a category of ambulances based at least in part on the assessed health condition of the patient. The method further includes sending a first alert to one or more ambulances of the selected category of ambulances, the first alert comprising the location of the patient, and one or more hospitals from the selected category of hospitals.

In another embodiment of the present disclosure, the method includes receiving a profile of the patient, wherein the profile includes one or more of: (i) a physical profile of the patient, (ii) a gender profile of the patient, and (iii) medical history of the patient, wherein the physical profile comprises one or more of physique and age of the patient, the gender profile comprises gender of the patient, and the medical history of the patient comprises past health information of the patient. The method also includes receiving disaster-related information for the location, wherein the disaster-related information comprises information on one or more of: (i) epidemics in the location, (ii) hazards in the location, and (iii) weather and environmental conditions in the location, and wherein the health condition of the patient is assessed based at least in part on the emergency information, the profile of the patient, and the disaster related information.

In another embodiment of the present disclosure, the selection of the category of hospitals includes determining medical interventions required for the patient based at least in part on the assessed health condition of the patient and the profile of the patient, where the medical interventions include: surgery, treatment, therapy, or a combination thereof. The method also includes determining the time within which the determined medical interventions need to be provided to the patient, determining equipment required for the determined medical interventions, and determining healthcare professionals required for the determined medical interventions and the determined equipment. The method further includes identifying the category of hospitals from a plurality of categories of hospitals, based on the determined medical interventions, the determined time, the determined equipment, and the determined healthcare professionals.

In yet another embodiment of the present disclosure, the selection of the category of ambulances includes determining on-board medical facilities required for the patient in a desired ambulance based at least in part on the determined time and the determined medical interventions, determining on-board equipment required in the desired ambulance based at least in part on the determined medical facilities and the physical profile of the patient, determining on-board healthcare professionals required for the determined medical facilities and the determined equipment, and identifying the category of ambulances from a plurality of categories of ambulances, based on the determined on-board medical facilities, the determined on-board equipment, and the determined on-board healthcare professionals.

In an additional embodiment of the present disclosure, the one or more ambulances of the selected category of ambulances are determined based at least in part on factors including locations of ambulances of the selected category of ambulances, available routes from the respective locations of the ambulances to the location of the patient, estimated weather conditions on the available routes, estimated wait times at traffic signals on the available routes, estimated traffic conditions on the available routes, estimated travel times from the respective locations of the ambulances to the location of the patient, estimated VIP movement on the available routes, and estimated public events in the vicinity of the available routes.

In a further embodiment of the present disclosure, the one or more hospitals are determined based at least in part on factors including locations of hospitals of the selected category of hospitals, available routes from the location of the patient to the respective locations of the hospitals, estimated weather conditions on the available routes, estimated wait times at traffic signals on the available routes, estimated traffic conditions on the available routes, estimated travel times from the location of the patient to the respective locations of the hospitals, estimated VIP movement on the available routes, and estimated public events in the vicinity of the available routes.

In another embodiment of the present disclosure, a first category of hospitals is selected when the assessed health condition is a first health condition, and a second category of hospitals is selected when the assessed health condition is a second health condition. Further, a first category of ambulances is selected when the assessed health condition is the first health condition and the profile of the patient is a first profile, and a second category of ambulances is selected when the assessed health condition is the first health condition and the profile of the patient is a second profile.

In yet another embodiment of the present disclosure, the system stores a corresponding record for each subscriber of emergency response services, wherein a record includes unique identifier of a corresponding subscriber and profile of the corresponding subscriber, wherein the profile of the corresponding subscriber includes one or more of: physical profile of the corresponding subscriber, gender profile of the corresponding subscriber, and medical history of the corresponding subscriber.

In a further embodiment, the patient is a subscriber, and the method further includes receiving a unique identifier corresponding to the patient from a user device, where the unique identifier is one of an alphanumeric code, a numeric code and a QR code. The method also includes prompting a user of the user device to confirm that the subscriber is in an emergency situation at the location from which the unique identifier is sent, receiving a confirmation from the user device, determining a location of the user device upon receiving the confirmation, and prompting the user to send emergency information related to the subscriber. The emergency information related to the patient is received in response to the prompting of the user to send the emergency information related to the subscriber, the determining of the location of the patient comprises determining the location of user device as the location of patient, and the receiving of the profile of the patient includes retrieving the profile of the patient from corresponding record of the patient using the unique identifier corresponding to the patient.

In another embodiment, the record corresponding to the patient also includes one or more emergency contacts of the patient; and at least one of (i) medical insurance details of the patient, wherein the medical insurance details comprise details of one or more medical insurance accounts linked to the patient, and (ii) details of one or more digital wallets of the patient, wherein the one or more digital wallets contain funds for emergency services. Where, the method further includes sending a second alert to a hospital, the second alert including the present health condition of the patient, and sending a communication to the one or more emergency contacts of the patient, where the communication includes one or more of: real-time location of the subscriber, real-time location of the ambulance, location of the hospital, contact details of the ambulance, and contact details of the hospital. The method also includes guiding the ambulance to the hospital, and facilitating automated registration of the patient with the hospital using the unique identifier of the patient and one of (i) a medical insurance account of the patient, and (ii) a digital wallet of the patient.

In yet another embodiment, the health condition of the patient is assessed using a first multimodal machine-learning model. Inputs of the first multimodal machine-learning model include the emergency information related to the patient, the profile of the patient, and the disaster related information for the location. The inputted emergency information includes at least one of (a) the description of the injuries of the patient, and (b) the description of the health issues of the patient; and the visual media depicting present state of the patient. The inputted profile of the patient input includes the physical profile of the patient, the gender profile of the patient, and the medical history of the patient. The inputted disaster related information for the location includes epidemics in the location, hazards in the location, and weather and environmental conditions in the location. One or more outputs of the multimodal machine-learning model include the assessed health condition of the patient.

Another aspect of the present disclosure provides for a system for managing emergency response services, the system includes a memory to store instructions, and one or more processors to execute the instructions stored in the memory to cause the system to perform the actions of receiving emergency information related to a patient, where the emergency information contains one or more of: description of injuries of the patient, description of health issues of the patient, and visual media depicting present state of the patient. The actions also include determining a location of the patient, assessing health condition of the patient based at least in part on the received emergency information, and selecting a category of hospitals and a category of ambulances based at least in part on the assessed health condition of the patient. The actions further include sending a first alert to one or more ambulances of the selected category of ambulances, the first alert comprising the location of the patient, and one or more hospitals from the selected category of hospitals.

In another embodiment of the present disclosure, the system is to perform the actions of receiving a profile of the patient, wherein the profile includes one or more of: (i) a physical profile of the patient, (ii) a gender profile of the patient, and (iii) medical history of the patient, wherein the physical profile comprises one or more of physique and age of the patient, the gender profile comprises gender of the patient, and the medical history of the patient comprises past health information of the patient. The actions also include receiving disaster-related information for the location, wherein the disaster-related information comprises information on one or more of: (i) epidemics in the location, (ii) hazards in the location, and (iii) weather and environmental conditions in the location, and wherein the health condition of the patient is assessed based at least in part on the emergency information, the profile of the patient, and the disaster related information.

In another embodiment of the present disclosure, the selection of the category of hospitals includes determining medical interventions required for the patient based at least in part on the assessed health condition of the patient and the profile of the patient, where the medical interventions include: surgery, treatment, therapy, or a combination thereof. The system is to perform the actions including determining the time within which the determined medical interventions need to be provided to the patient, determining equipment required for the determined medical interventions, and determining healthcare professionals required for the determined medical interventions and the determined equipment. The actions further include identifying the category of hospitals from a plurality of categories of hospitals, based on the determined medical interventions, the determined time, the determined equipment, and the determined healthcare professionals.

In yet another embodiment of the present disclosure, the selection of the category of ambulances includes determining on-board medical facilities required for the patient in a desired ambulance based at least in part on the determined time and the determined medical interventions, determining on-board equipment required in the desired ambulance based at least in part on the determined medical facilities and the physical profile of the patient, determining on-board healthcare professionals required for the determined medical facilities and the determined equipment, and identifying the category of ambulances from a plurality of categories of ambulances, based on the determined on-board medical facilities, the determined on-board equipment, and the determined on-board healthcare professionals.

In an additional embodiment of the present disclosure, the one or more ambulances of the selected category of ambulances are determined based at least in part on factors including locations of ambulances of the selected category of ambulances, available routes from the respective locations of the ambulances to the location of the patient, estimated weather conditions on the available routes, estimated wait times at traffic signals on the available routes, estimated traffic conditions on the available routes, estimated travel times from the respective locations of the ambulances to the location of the patient, estimated VIP movement on the available routes, and estimated public events in the vicinity of the available routes.

In a further embodiment of the present disclosure, the one or more hospitals are determined based at least in part on factors including locations of hospitals of the selected category of hospitals, available routes from the location of the patient to the respective locations of the hospitals, estimated weather conditions on the available routes, estimated wait times at traffic signals on the available routes, estimated traffic conditions on the available routes, estimated travel times from the location of the patient to the respective locations of the hospitals, estimated VIP movement on the available routes, and estimated public events in the vicinity of the available routes.

In yet another embodiment, the health condition of the patient is assessed using a first multimodal machine-learning model. Inputs of the first multimodal machine-learning model include the emergency information related to the patient, the profile of the patient, and the disaster related information for the location. The inputted emergency information includes at least one of (a) the description of the injuries of the patient, and (b) the description of the health issues of the patient; and the visual media depicting present state of the patient. The inputted profile of the patient input includes the physical profile of the patient, the gender profile of the patient, and the medical history of the patient. The inputted disaster related information for the location includes epidemics in the location, hazards in the location, and weather and environmental conditions in the location. One or more outputs of the multimodal machine-learning model include the assessed health condition of the patient.

In another embodiment of the present disclosure, a first category of hospitals is selected when the assessed health condition is a first health condition, and a second category of hospitals is selected when the assessed health condition is a second health condition. Further, a first category of ambulances is selected when the assessed health condition is the first health condition and the profile of the patient is a first profile, and a second category of ambulances is selected when the assessed health condition is the first health condition and the profile of the patient is a second profile.

In a further embodiment of the present disclosure, the system stores a corresponding record for each subscriber of emergency response services, wherein a record includes unique identifier of a corresponding subscriber and profile of the corresponding subscriber, wherein the profile of the corresponding subscriber includes one or more of: physical profile of the corresponding subscriber, gender profile of the corresponding subscriber, and medical history of the corresponding subscriber. When the patient is a subscriber, and the system is to further perform the actions including receiving a unique identifier corresponding to the patient from a user device, where the unique identifier is one of an alphanumeric code, a numeric code and a QR code. The actions also include prompting a user of the user device to confirm that the subscriber is in an emergency situation at the location from which the unique identifier is sent, receiving a confirmation from the user device, determining a location of the user device upon receiving the confirmation, and prompting the user to send emergency information related to the subscriber. The emergency information related to the patient is received in response to the prompting of the user to send the emergency information related to the subscriber, the determining of the location of the patient comprises determining the location of user device as the location of patient, and the receiving of the profile of the patient includes retrieving the profile of the patient from corresponding record of the patient using the unique identifier corresponding to the patient. The record corresponding to the patient also includes one or more emergency contacts of the patient; and at least one of (i) medical insurance details of the patient, wherein the medical insurance details comprise details of one or more medical insurance accounts linked to the patient, and (ii) details of one or more digital wallets of the patient, wherein the one or more digital wallets contain funds for emergency services. Where, the actions also include sending a second alert to a hospital, the second alert including the present health condition of the patient, and sending a communication to the one or more emergency contacts of the patient, where the communication includes one or more of: real-time location of the subscriber, real-time location of the ambulance, location of the hospital, contact details of the ambulance, and contact details of the hospital. The actions also include guiding the ambulance to the hospital, and facilitating automated registration of the patient with the hospital using the unique identifier of the patient and one of (i) a medical insurance account of the patient, and (ii) a digital wallet of the patient.

These and other embodiments of the present disclosure are discussed in further detail hereinbelow.

### BRIEF DESCRIPTION OF THE FIGURES

These and other features, aspects, and advantages of the example embodiments will become better understood when the following detailed description is read with reference to the accompanying drawings in which like characters represent like parts throughout the drawings, wherein:

FIG. 1 depicts an example environment in which various aspects of the present disclosure are implemented;

FIG. 2 is a flowchart illustrating a method for managing emergency response services, according to the aspects of the present disclosure;

FIGS. 3A and 3B is a flowchart illustrating a method for managing emergency response services, in an embodiment of the present disclosure.

FIG. 4A and FIG. 4B depict example implementation environments of the aspects of the present disclosure.

FIG. 5 depicts an example record corresponding to a subscriber, according to aspects of the present disclosure.

FIG. 6 illustrates an example mapping of a record to a QR code, according to aspects of the present disclosure.

FIG. 7 depicts a block diagram of ERS management system, according to an example implementation of the present disclosure.

FIG. 8 is a block diagram illustrating the details of digital processing system in which various aspects of the present disclosure are operative by execution of appropriate executable modules.

### DETAILED DESCRIPTION OF EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in many alternate forms and should not be construed as limited to only the example embodiments set forth herein.

Accordingly, while example embodiments are capable of various modifications and alternative forms, example embodiments are shown by way of example in the drawings and will herein be described in detail. It should be understood, however, that there is no intent to limit example embodiments to the particular forms disclosed. On the contrary, example embodiments are to cover all modifications, equivalents, and alternatives thereof. Similarly, like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed but may also have additional steps not included in the figure. The processes may correspond to methods, functions, procedures, subroutines, subprograms, etc.

Specific structural and functional details disclosed herein are merely representative for purposes of describing example embodiments. Inventive concepts may, however, be embodied in many alternate forms and should not be construed as limited to only the example embodiments set forth herein.

It will be understood that, although the terms first, second, etc. may be used herein to describe various elements, these elements should not be limited by these terms. These terms are only used to distinguish one element from another. For example, a first element could be termed a second element, and similarly, a second element could be termed a first element, without departing from the scope of example embodiments. As used herein, the term “and/or” includes any, and all combinations of one or more of the associated listed items. The phrase “at least one of” has the same meaning as “and/or”.

Further, although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the scope of inventive concepts.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a fashion (e.g., “between,” versus “directly between,” “adjacent,” versus “directly adjacent,” etc.).

The terminology used herein is for the purpose of describing particular example embodiments only and is not intended to be limiting. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, elements, and/or components, but do not preclude the presence or addition of one or more other features, integers, steps, operations, elements, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

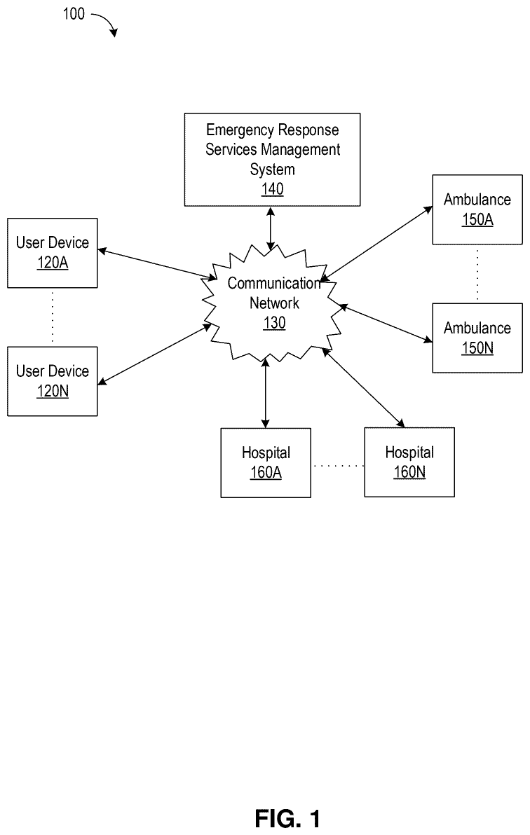

FIG. 1 is a block diagram illustrating example environment 100 in which various aspects of the present disclosure are implemented. Example environment 100 is shown containing user devices 120A-120N, communication network 130, emergency response services (ERS) management system 140, ambulances 150A-150N, and hospitals 160A-160N. The block diagram is shown with a representative set of blocks only for illustration, and it is understood that environments employing aspects of the present disclosure can also have other types and number of blocks.

For the sake of conciseness, in the present disclosure, user devices 120A-120N are also referred to as user device 120, ambulances 150A-150N are also referred to as ambulance 150, and hospital 160A-160N are also referred to as hospital 160. Each block of FIG. 1 is described below in detail.

Each user device 120A-120N is a device capable of sending/receiving communications. User device 120 may send/receive communications using protocols and/or techniques known in the relevant arts. In an embodiment, user device 120 sends/receives communications to/from ERS management system 140. The communications may include, but not limited to, requests for emergency services sent from user device 120, requests for information/confirmation pertaining to emergency situations received at user device 120, information/confirmation provided from user device 120, information/confirmation received at user device 120, etc.

User device 120 may also be capable of running various software applications. In an embodiment, user device 120 communicates with ERS management system 140, and other blocks in FIG. 1 using a software application. In addition, user device 120 may contain a camera, and may be capable of taking photographs, videos and scanning images (for example, QR codes, barcodes, etc.).

In an example implementation, user device 120 is a mobile device (for example, a mobile phone, a tablet, a laptop, etc.). However, in alternative implementations, user device 120 can be any device known in the relevant arts.

Communication network 130 is a network capable of providing connectivity and communications among various blocks of FIG. 1. Communication network 130 may be implemented using protocols/techniques, well known in the relevant arts. For example, communication network 130 may be a telephone network, data network, or a converged network supporting voice, data, video, VoIP, and internet communications.

Each ambulance 150A-150N is a vehicle equipped to transport patients to hospitals, and to provide on-board medical facilities to the patients during transit. Each ambulance 150 may contain equipment necessary to provide such medical facilities and facilitate patient mobility, including equipment for safe boarding and deboarding of the patients. Additionally, each ambulance may be staffed with suitably trained healthcare professionals (such as paramedics, doctors, on-board equipment operators, etc.) to provide the medical facilities and to operate the equipment.

In an embodiment, ambulances 150A-150N include different categories, where each category is suitably equipped to provide specific type of on-board medical facilities and/or transportation service, and is staffed with healthcare professionals suitably trained in providing the corresponding type of medical facilities and/or transportation.

In an example embodiment, ambulances are classified into different categories as below:
(i) Basic Life Support (BLS) ambulances to provide basic medical facilities, equipped with basic equipment such as oxygen cylinders, first aid kits, stretchers, and bandages, and staffed with healthcare professionals trained to provide the basic medical facilities and to operate the on-board equipment.
(ii) Advanced Life Support (ALS) ambulances to provide critical care medical facilities, equipped with defibrillators, ventilators, intravenous (IV) supplies and medications, and staffed with healthcare professionals trained to provide the critical care medical facilities and to operate the on-board equipment.
(iii) Pediatric ambulances to provide pediatric medical facilities, equipped with incubators, child-sized ventilators and monitors, and staffed with healthcare professionals trained to provide pediatric or neonatal care and to operate the on-board equipment.
(iv) Trauma ambulances to provide physical trauma care medical facilities, equipped with immobilization such as spine boards and cervical collars, trauma kits for managing severe bleeding and medications for pain relief, and staffed with healthcare professionals trained to provide the trauma care medical facilities and to operate the on-board equipment.
(v) Cardiac ambulances to provide cardiac care medical facilities, equipped with ECG monitors and defibrillators, and staffed with healthcare professionals trained to provide cardiac care medical facilities and to operate the on-board equipment.
(vi) Bariatric ambulances to transport obese patients, equipped with wider stretchers and hydraulic lifts, and staffed with healthcare professionals trained to provide required medical facilities and to operate the on-board equipment.
(vii) Maternity ambulances to provide obstetric emergency care medical facilities, equipped with fetal monitors and oxygen supplies, and staffed with healthcare professionals trained to provide obstetric emergency care medical facilities and to operate the on-board equipment.
(viii) ICU ambulances to provide intensive care medical facilities, equipped with ventilators, infusion pumps and multi-parameter monitors, and staffed with healthcare professionals trained to provide the intensive care medical facilities and to operate the on-board equipment.
(ix) Air ambulances (such as helicopters or fixed-wing aircrafts) to airlift patients, equipped to provide ICU-level care, and staffed with suitably trained healthcare professionals.
(x) Mortuary Ambulances equipped with cooling systems for respectful and secure transport of deceased patients.
(xi) Special Transport ambulances equipped with comfortable stretchers or seating arrangements and basic medical supplies, to transport bedridden patients or those requiring assistance during travel in non-emergency situations.
   It is understood that, in alternative embodiments, other categories of ambulances may also exist. Also, some of the above noted categories may be further categorized based on one or more of physique, age, and gender of the patients.

Each hospital 160A-160N is equipped to provide medical interventions to the patients. The medical interventions include treatment, surgery, medication, or any other procedures, including a combination thereof. Each hospital 160 is also furnished with the necessary equipment to provide the medical interventions, and is staffed with healthcare professionals trained to provide such medical interventions and to operate the equipment.

In an embodiment, hospitals 160A-160N are classified into different categories based on one or more factors such as the medical interventions provided, available equipment, and qualifications of healthcare professionals. In another embodiment, additional patient demographic factors, including physique, age, and/or gender, are also considered when categorizing hospitals 160A-160N.

ERS management system 140 manages emergency response services. ERS management system 140 is capable of communicating (including sending/receiving communications) with user devices 120A-120N, ambulances 150A-150N and hospitals 160A-160N, using techniques known in the relevant arts.

ERS management system 140 is also capable of storing and retrieving details of subscribers to emergency response services, ambulances 150A-150N, and hospitals 160A-160N in a database associated with ERS management system 140, using techniques known in the relevant arts.

In an embodiment, ERS management system 140 stores the details of ambulances 150A-150N as per the categories of the ambulances, and the details of hospitals 160A-160N as per the categories of the hospitals.

ERS management system 140 is further capable of determining and tracking locations of user devices 120A-120N and ambulances 150A-150N, and storing location details of hospitals 160A-160N. ERS management system 140 may use techniques known in the relevant arts for this purpose.

ERS management system 140 is also capable of suggesting ambulances and hospitals to requesters of emergency response services, and guide ambulances to requesters of emergency response services (for example, patients) and to hospitals. ERS management system 140 may use techniques known in the relevant arts for this purpose.

ERS management system 140 may also use various geo-location techniques, known in the relevant arts, to determine the locations of users 120A-120N and ambulances 150A-150A. For example, location of user device 120 can be determined based on (i) GPS coordinates embedded in metadata of the visual media received, (ii) IP address of user device 120 from which the information related to an emergency situation is received, (iii) location data obtained from device-originated emergency alerts, such as those generated via a mobile application in user device 120 or a wearable health device worn by a patient; (iv) network-based location heuristics, including but not limited to cell tower triangulation and Wi-Fi positioning; and (v) previously registered location information associated with user device 120 in the ERS management system 140. Similarly, the locations of ambulances 150A-150N may also be determined using various geo-location techniques known in the relevant arts.

Also, ERS management system 140 may use, routing techniques known in the relevant arts, to route ambulances 150A-150N to the patients and to the hospitals 160A-160N.

In addition, ERS management system 140 is capable of processing multimedia (including text, audio, video, and images). ERS management system 140 may use techniques known in the relevant art for this purpose.

Further, ERS management system 140 is capable of running various software applications and machine learning models. In an embodiment, ERS management system 140 runs various software applications and/or machine learning models to process information, multimedia (including text, audio, video, and images (including QR codes and barcodes) and data received from various sources (such as internet, sensors, other devices/systems). In an embodiment, ERS management system 140 also runs various software applications and/or machine learning models to classify ambulances and hospitals into various categories, suggest ambulances and hospitals to requesters of emergency response services, to guide ambulances to the requesters of emergency response services (for example, patients) and to the hospitals, and to send alerts to the ambulances 150 and hospitals 160.

In an example implementation, ERS management system 140 is a remote server configured on a cloud platform. However, in alternative implementations, ERS management system 140 can be implemented using any computing/processing system known in the relevant arts.

Though not shown in FIG. 1, example environment 100 may also contain emergency contacts of the patients, police, government authorities, etc., and ERS management system 140 may communicate with the same.

The description is continued with respect to flowcharts illustrating the manner in which emergency response services are managed according to the aspects of the present disclosure.

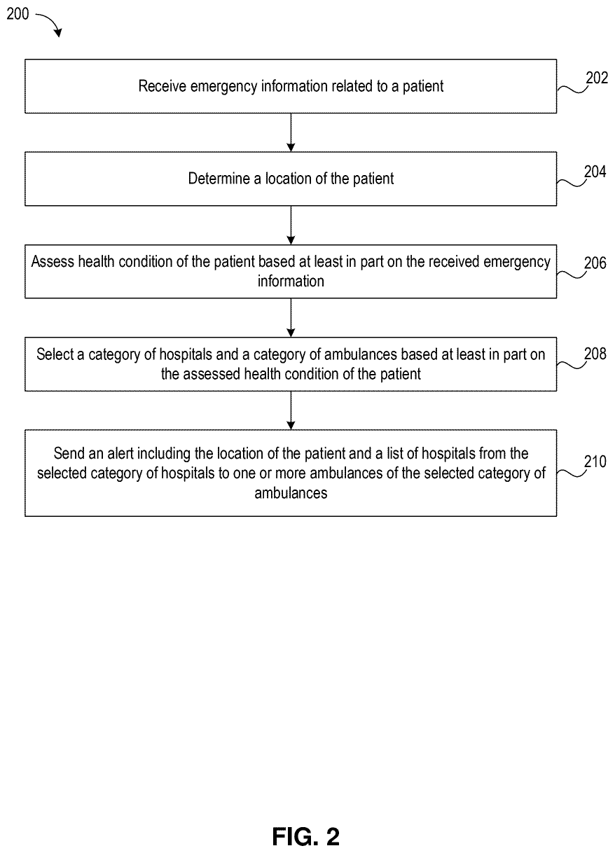

FIG. 2 is a flowchart illustrating method 200 for managing emergency response services, according to the aspects of the present disclosure. The features of FIG. 2 are described with respect to FIG. 1, and the steps of the flowchart of FIG. 2 are described as being performed at ERS management system 140 only for illustration. However, the steps can be performed in other environments and systems as will be apparent to a skilled person.

In step 202, ERS management system 140 receives emergency information related to a patient. The emergency information includes one or more of: (i) description of injuries of the patient, (ii) description of health issues of the patient, and (iii) visual media depicting present state of the patient. An injury is a tangible physical damage to the body of the patient. A health issue refers to an illness, disease, chronic condition, disorder, or a medical problem. The description of the injuries of the patient and the description of the health issues of the patient can be in the form of text or audio. The visual media refers to visual representations capturing the present situation of the patient (such as physical appearance, injuries, posture, environment, visible symptoms and trauma related assessment). The visual media can be in the form of image(s) or video(s). In an embodiment, the emergency information may also contain location details of the patient.

In an embodiment, the emergency information is received from user device 120. User device 120 may correspond to the user device of the patient or the user device of a bystander.

In step 204, ERS management system 140 determines a location of the patient. If the received emergency information contains location details of the patient, ERS management system 140 determines the location of the patient by processing the location details in the received emergency information. If the location details are not contained in the received emergency information, ERS management system 140 determines the location of the patient using geo-location techniques well known in the relevant arts, for example, based on (i) GPS coordinates embedded in metadata of the visual media received, (ii) IP address of user device 120 from which the information related to the emergency situation is received, (iii) location data obtained from device-originated emergency alerts, such as those generated via a mobile application in user device 120; (iv) network-based location heuristics, including but not limited to cell tower triangulation and Wi-Fi positioning; and (v) previously registered location information associated with user device 120 in the ERS management system 140.

In step 206, ERS management system 140 assesses health condition of the patient based at least in part on the received emergency information. In an embodiment, ERS management system 140 assesses the health condition of the patient by processing the received emergency information. The received emergency information can be processed either by using the techniques known in the relevant arts or by using the techniques disclosed in the present disclosure.

In an embodiment, ERS management system 140 also receives a profile of the patient and disaster-related information for the location, and assesses the health condition of the patient based at least in part on the emergency information, the profile of the patient, and the disaster related information.

The profile of the patient includes one or more of (i) a physical profile of the patient, (ii) a gender profile of the patient, and (iii) medical history of the patient. The physical profile includes physique and/or age of the patient, the gender profile includes gender of the patient, and the medical history of the patient includes past health information of the patient. The disaster-related information for the location includes information on one or more of: (i) epidemics in the location, (ii) hazards in the location, and (iii) weather and environmental conditions in the location.

In an example implementation, ERS management system 140 assesses the health condition of the patient using one or more machine learning model(s) that are suitably trained.

In step 208, ERS management system 140 selects a category of hospitals and a category of ambulances, based at least in part, on the assessed health condition of the patient. In an embodiment, ERS management system 140 also takes into account the profile of the patient in addition to the assessed health condition in selecting the category of hospitals and the category of ambulances. ERS management system 140 may also take other factors into consideration for the selection of the category of hospitals and the category of ambulances.

In an embodiment, the selecting of the category of hospitals includes ERS management system 140 determining medical interventions (including surgery, treatment, therapy, or a combination thereof) required for the patient based at least in part on the assessed health condition of the patient and the profile of the patient, the time within which the determined medical interventions need to be provided to the patient, equipment required for the medical interventions, healthcare professionals required for the determined required medical interventions. ERS management system 140 identifies (selects) the category of hospitals from multiple categories of hospitals, based on the determined medical interventions, the determined time, the determined equipment, and the determined healthcare professionals.

Also, in an embodiment, the selecting of the category of ambulances includes ERS management system 140 determining on-board medical facilities required for the patient in a desired ambulance based at least in part on the determined time and the determined medical interventions, on-board equipment required in the desired ambulance based at least in part on the determined medical facilities and the physical profile of the patient, and on-board healthcare professionals required for the determined medical facilities and the determined equipment. ERS management system 140 identifies (selects) the category of ambulances from multiple categories of ambulances, based on the determined on-board medical facilities, the determined on-board equipment, and the determined on-board healthcare professionals.

In step 210, ERS management system 140 sends an alert (a first alert) to one or more ambulances of the selected category of ambulances. In an embodiment, the alert contains the location of the patient, and one or more hospitals from the selected category of hospitals.

Thus, method 200 operates to select different categories of hospitals based on the assessed health condition i.e., a first category of hospitals is selected when the assessed health condition is a first health condition, and a second category of hospitals is selected when the assessed health condition is a second health condition. Also, method 200 operates to select different categories of ambulances for the same health condition, based on the profile of the patient i.e., a first category of ambulances is selected when the assessed health condition is the first health condition and the profile of the patient is a first profile, and a second category of ambulances is selected when the assessed health condition is the first health condition and the profile of the patient is a second profile.

Also, method 200 has several advantages. For instance, the method improves accuracy in assessing the health condition of the patient, by integrating diverse inputs, such as, emergency information, patient profile, and local disaster-related data. Also, the method facilitates tailored selection of hospitals and ambulances by taking into consideration the assessed health condition of the patient and the profile of the patient. Further, the method selects hospital and ambulance categories based on medical interventions, time sensitivity, equipment, availability of healthcare professionals, and personnel needs, thus optimizing response effectiveness.

The description is continued below with respect to the manner in which emergency responses are managed in an embodiment of the present disclosure.

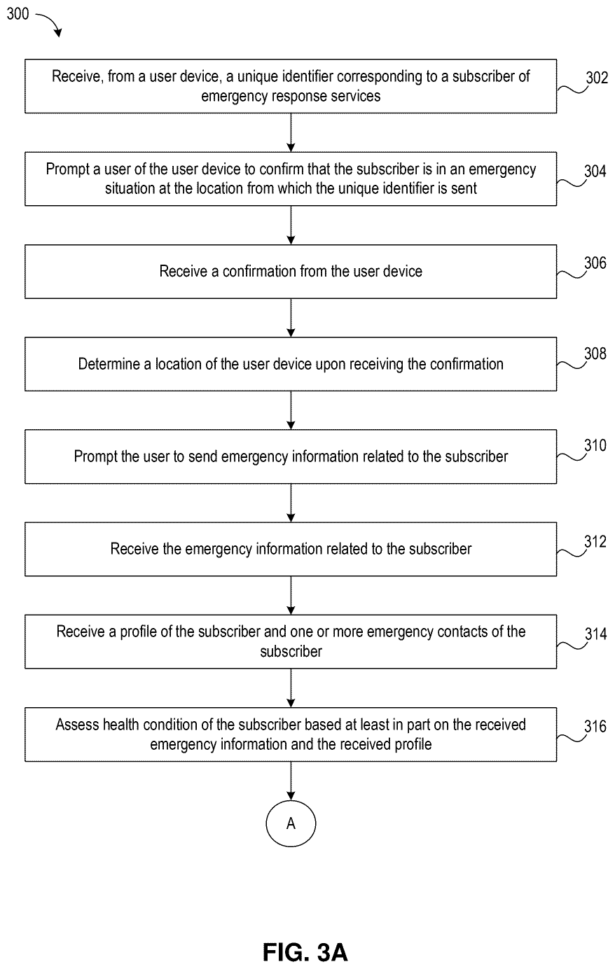

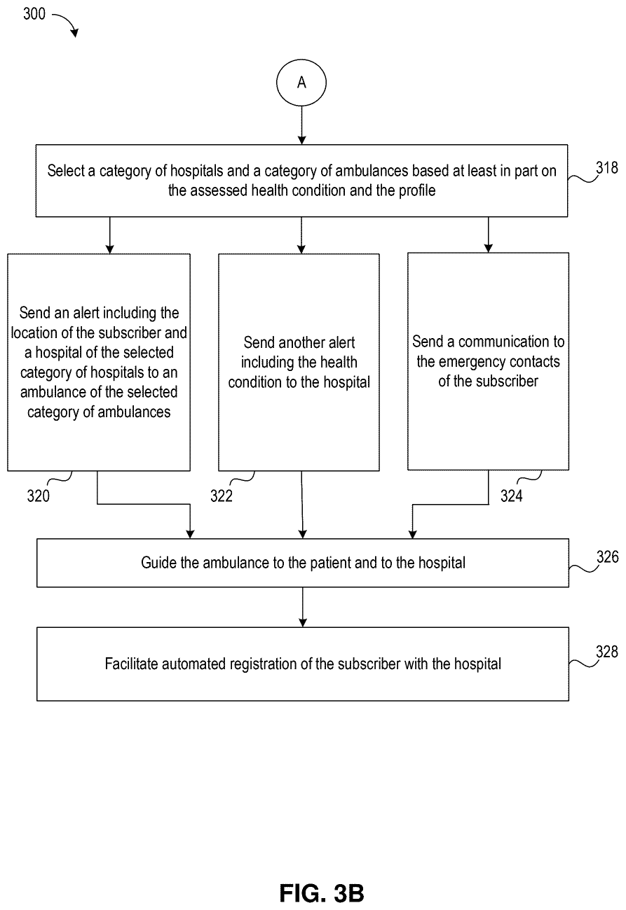

FIGS. 3A and 3B is a flowchart illustrating a method for managing emergency response services, in an embodiment of the present disclosure, where the patient is a subscriber of ERS management system 140 for emergency response services. The features of FIG. 3 are described with respect to FIG. 1, and the steps of the flowchart of FIG. 3 are described as being performed at ERS management system 140 only for illustration. However, the steps can be performed in other environments and systems as will be apparent to a skilled person.

In step 302, ERS management system 140 receives a unique identifier corresponding to a subscriber of emergency response services, from user device 120. In an embodiment, the unique identifier is an alphanumeric code, or a numeric code, or QR code. However, in alternative embodiments, the unique identifier can be in any other form as will be apparent to a skilled person. User device 120 may correspond to the user device of the patient or the user device of a bystander. In an example implementation, user device 120 is a mobile device (for example, a mobile phone, a tablet, a laptop, etc.). However, in alternative implementations, user device 120 can be any device known in the relevant arts.

In step 304, ERS management system 140 prompts a user of user device 120 to confirm that the subscriber is in an emergency situation at the location from which the unique identifier is sent. Techniques known in the relevant art may be used for such prompting. For example, the prompts can include: (i) text messaging/SMS prompts that request the user to confirm the emergency situation, where reply texts such as “Yes,” “No,” or numeric codes serve as confirmation or denial; (ii) automated voice calls or voice prompts that require the user to confirm the emergency, with responses provided verbally; and (iii) software/mobile application notifications that push messages via emergency response or health apps, allowing the user to tap or respond by confirming or requesting help. However, alternative techniques known in the relevant arts may also be used for such prompting.

In step 306, ERS management system 140 receives a confirmation from user device 120, indicating that the user is in fact in an emergency situation at the location from which the unique identifier is sent.

In step 308, ERS management system 140 determines a location of user device 120 upon receiving the confirmation. The location of user device 120 can be determined using a geo-location technique known in the relevant art. For example, the location of user device 120 can be determined based on (i) IP address of user device 120, (ii) location data obtained from device-originated emergency alerts, such as those generated via a mobile application in user device 120, (iii) network-based location heuristics, including but not limited to cell tower triangulation and Wi-Fi positioning, and (iv) previously registered location information associated with user device 120 in the ERS management system 140.

In step 310, ERS management system 140 prompts the user to send emergency information related to the subscriber. In step 312, ERS management system 140 receives the emergency information related to the subscriber. The prompt can be in any form as known in the relevant arts, including the example forms noted above in step 304. Also, the emergency information can contain contents as explained above with respect to FIG. 2.

In step 314, ERS management system 140 receives profile of the subscriber and one or more emergency contacts of the subscriber. In an embodiment, receiving the profile and the one or more emergency contacts includes retrieving the profile and the emergency contacts from the database associated with ERS management system 140 using the unique identifier of the subscriber. The profile of the subscriber can contain contents as explained above with respect to FIG. 2. Emergency contacts of the subscriber include the contact numbers of the designated persons who are to be notified if the subscriber is in an emergency situation.

In step 316, ERS management system 140 assesses health condition of the subscriber based at least in part on the received emergency information and the received profile, as explained above with respect to FIG. 2.

In step 318, ERS management system 140 selects a category of hospitals and a category of ambulances based at least in part on the assessed health condition and the profile, as noted above with respect to the description of FIG. 2.

In step 320, ERS management system 140 sends an alert including the location of the subscriber and a hospital of the selected category of hospitals to an ambulance of the selected category of ambulances.

In an embodiment, the hospital is identified (selected) from the selected category of hospitals based at least in part on factors, including but not limited to, (i) locations of hospitals of the selected category of hospitals; (ii) available routes from the location of the patient to the respective locations of the hospitals; (iii) estimated weather conditions on the available routes; (iv) estimated wait times at traffic signals on the available routes; (v) estimated traffic conditions on the available routes; (vi) estimated travel times from the location of the patient to the respective locations of the hospitals; (vii) estimated VIP movement on the available routes; and (viii) estimated public events in the vicinity of the available routes.

In an embodiment, the ambulance is identified (selected) from the selected category of ambulances based at least in part on factors, including but not limited to, (i) locations of ambulances of the selected category of ambulances; (ii) available routes from the respective locations of the ambulances to the location of the patient; (iii) estimated weather conditions on the available routes; (iv) estimated wait times at traffic signals on the available routes; (v) estimated traffic conditions on the available routes; (vi) estimated travel times from the respective locations of the ambulances to the location of the patient; (vii) estimated VIP movement on the available routes; and (viii) estimated public events in the vicinity of the available routes.

VIP movement refer to estimated transit or travel patterns of Very Important Persons (VIPs) on the available routes. VIP movement typically involves managed or escorted travel with potential roadblocks, traffic redirections, or special traffic privileges to ensure their timely and secure passage. Public events refer to gatherings or occasions held in public spaces that can impact traffic conditions and ambulance routing. These events include assemblies, parades, celebrations, concerts, sports events, festivals, and other mass gatherings that may cause road closures, diversions, or increased congestion in the vicinity of ambulance routes.

In step 322, ERS management system 140 sends another alert (a second alert) to the hospital, where the second alert includes the health condition of the patient.

In step 324, ERS management system 140 sends a communication to one or more emergency contacts of the subscriber. The communication includes one or more of (i) real-time location of the subscriber, (ii) real-time location of the ambulance, (iii) location of the hospital, (iv) contact details of the ambulance, and (v) contact details of the hospital.

In step 326, ERS management system 140 guides the ambulance to the patient and to the hospital. Routing techniques known in the relevant art may be used for the purpose of guiding the ambulance to the hospital. For example, the routing techniques can include, GPS-based navigation, real-time traffic data analysis, shortest path calculation, and route optimization techniques to minimize travel time. In an example implementation, ERS management system 140 uses one or more machine learning models for guiding the ambulance to the hospital.

In step 328, ERS management system 140 facilitates automated registration of the subscriber with the hospital. In an embodiment, ERS management system 140 facilitates the automated registration using the unique identifier of the patient, and one of (i) a medical insurance account of the patient, and (ii) a digital wallet of the patient which contains funds for emergency services, retrieved from the record associated with the subscriber in the database associated with ERS management system 140.

Though FIG. 3 illustrates steps 320, 322 and 324 as being performed simultaneously, in alternative embodiments, the steps may be performed sequentially too.

Also, in alternative embodiments, the steps of FIG. 3 may be performed in a different sequence.

The method of FIG. 3 has several advantages. For instance, the method seamlessly integrates various steps in emergency response services, such as, identification of the patient, confirmation/verification of the emergency situation, tracking location of the patient, health assessment, tailor-made determination of the ambulance and the hospital, sending alerts and communication, navigation of the ambulance to the patient and then to the hospital, and automation of registration with the hospital. Such seamless integration enhances speed, reliability and effectiveness of emergency responses.

Specifically, the use of a unique identifier of the subscriber facilitates fast, precise and accurate retrieval of patient's details from the database. The dynamic selection of hospital and ambulance categories based on the assessed health condition and the profile optimizes resource allocation according to severity and needs. Consideration of the real-time factors (such as traffic, weather, VIP movement, public events, etc.) in hospital and ambulance selection enhances the efficiency and timeliness of emergency response. Communication to hospitals and emergency contacts with real-time location and condition information improves coordination and preparedness. Automated hospital registration and billing using the subscriber identifier, and the insurance account/digital wallet simplifies administrative processes and speeds patient's admission into the hospital.

The description is continued below with respect to the manner in which ERS management system 140 assesses the health condition of the patient.

### Assessing Health Condition of a Patient

ERS management system 140 provides multiple heterogeneous data streams as inputs to a first multimodal machine learning model. The data streams include:
(i) Emergency information related to the patient: This data includes textual descriptions including: (a) description of the patient's injuries (e.g., blunt force trauma, bleeding severity, fractures), and (b) description of other health issues (e.g., breathing difficulties, chest pain, unconsciousness). This data also includes visual media depicting the present state of the patient, such as photographs or real-time video frames captured at the scene, providing visual evidence of visible injuries, patient posture, skin color, or distress signs.
(ii) Profile of the patient: This data includes physical profile data including age, physique (such as height, weight, body mass index (BMI), and other biometric measurements), gender profile indicating biological sex or gender identity.
(iii) Medical history data: This data includes known chronic conditions, allergies, previous surgeries, current medications, and recent hospitalizations. This data may be retrieved from the patient's records in the database associated with ERS management system 140 or from the information provided by the patient.
(iv) Disaster-related information for the patient's location: This data includes epidemic events or outbreaks currently occurring in the geographical area (e.g., influenza, COVID-19), which may influence patient risk factors or medical treatment priority; and hazardous conditions present at the location (e.g., chemical spills, fires, structural damage), potentially impacting injury types or complicating care. This data also includes weather and environmental conditions affecting the location such as extreme heat, cold, air quality indices, or natural disaster aftermath (e.g., floods, storms).

All inputs are timestamped and geotagged where applicable to maintain spatio-temporal context and synchronization for accurate model inference.

The raw input data undergoes preprocessing and feature extraction to produce meaningful representations for machine learning analysis, as below:
(i) Textual emergency information: Natural Language Processing (NLP) techniques parse and tokenize injury and health issue descriptions, extracting keywords, symptom severity indicators, and contextual relationships. Entities and attributes are identified through named entity recognition (NER) and symptom classification models to standardize diverse narrative inputs.
(ii)Visual media: Images and video frames are processed via Computer Vision pipelines, extracting features such as color histograms, texture patterns, wound location and size, posture estimation, and skin pallor or cyanosis. Convolutional Neural Networks (CNNs) pre-trained on medical imaging datasets extract hierarchical visual features representing injury characteristics.
(iii) Patient profile data: Structured physical profile data, gender profile data, and medical history data are normalized, encoded, and embedded into numeric feature vectors suitable for downstream analysis. Medical history text notes or unstructured parts are further processed with NLP feature embedding techniques.
(iv) Disaster-related and Environmental data: Epidemic, hazard, and weather data are encoded as categorical or continuous indicators reflecting current levels of risk or environmental stress factors. These may be integrated as temporal sequences or static context vectors, depending on data availability.

Data cleaning, normalization, and alignment techniques may be performed to ensure consistent, noise-reduced multimodal feature vectors.

The multimodal machine learning model integrates features from all input modalities through the following architecture:

Separate neural network submodules are used to encode each input modality. Text encoders (e.g., transformer-based language models or LSTM networks) process emergency descriptions and medical history text. CNN-based visual encoders extract spatial features from patient images/videos. Fully connected networks or embedding layers encode structured patient profile and environmental data.

Encoded modality-specific features are concatenated or fused via attention mechanisms, gating layers, or multimodal transformers to capture inter-modality correlations, such as how visual injury severity corresponds with textual description or medical history. The fused representation is passed through further dense neural layers or recurrent architectures (e.g., LSTM) designed to model complex relationships and temporal dynamics between patient condition indicators and environmental factors.

Outputs of the machine-learning model include probabilistic or categorical assessments of the patient's health condition, such as severity scoring (e.g., minor, moderate, severe), triage categorization, or specific clinical condition labels (e.g., respiratory distress, haemorrhagic shock). Confidence scores and interpretable indicators (e.g., attention heatmaps on images or keywords) may also be provided for clinical validation.

The model is trained in a supervised manner using labelled datasets of emergency cases where the true assessed health condition is known. Data augmentation strategies for images and text balancing are employed to improve generalizability. Loss functions combine cross-entropy or mean squared error with possible multimodal consistency losses. Feedback loops incorporating new patient assessments, clinician validation, and outcome data can be used to improve performance and robustness in diverse contexts.

The description is continued below with respect to the manner in which ERS management system 140 selects the category of hospitals.

### Selection of the Category of Hospitals

ERS management system 140 provides multi-faceted inputs related to the patients'medical needs and profile as inputs to a second machine-learning model. The input modalities include:
(i) Assessed health condition: This includes outputs from the first machine learning modal, including severity scores, clinical condition labels, and triage categorizations.
(ii) Patient profile data: This includes age, gender, physique (height, weight, BMI), medical history (chronic diseases, allergies, previous surgeries), and other relevant physiological parameters.
(iii) Medical intervention data: This includes a knowledge base or ontology describing potential medical interventions such as surgery types, treatments, therapies, and combinations thereof, along with their requirements.
(iv) Temporal requirements: This includes typical or patient-specific clinically defined timelines within which medical interventions should be performed to optimize patient outcomes, sourced from medical guidelines or historical data.
(v) Equipment and resource requirements: This includes information on the medical devices, instruments, and therapeutic equipment needed to perform each type of intervention, including availability constraints.
(vi) Healthcare professional profiles: This includes data on the specialties, skills, certifications, and availability of healthcare providers capable of performing or assisting in the required interventions.
(vii) Hospital categorization database: This includes a repository specifying categories of hospitals (e.g., trauma centers, specialized surgical hospitals, rehabilitation centers), each defined by capabilities, resources, and staff qualifications.

Features extracted from the raw inputs include:
(i) Medical intervention determination features: Linking the assessed health condition and patient profile to required medical interventions by applying clinical rulesets, medical decision trees, or predictive models trained on historical intervention-outcome records; and encoding combinations or sequences of interventions if multiple treatments are indicated.
(ii) Intervention timing features: Extracting target intervention timeframes, expressed as time windows or deadlines for each intervention, based on patient condition urgency and clinical protocols.
(iii) Equipment requirements features: Mapping required interventions to equipment lists, specifying devices, instruments, consumables, and technology needed.
(iv) Healthcare professional requirements features: Matching required interventions and equipment to necessary healthcare professional qualifications, including surgeon specialties, nursing certifications, therapists, and support staff.
(v) Hospital capability encoding: Representing hospital categories and their capabilities as feature vectors, including presence of necessary equipment, staff qualifications, capacity to deliver interventions within required times, and historical success rates.

The above features in a modular machine learning framework:
(i) Medical intervention determination module: This module uses supervised classifiers or predictive decision models (e.g., gradient boosting trees, feed-forward neural networks) trained on labelled clinical cases to infer the interventions required for a given patient profile and assessed condition.
(ii) Timing and priority module: This module applies temporal modelling (e.g., deadline-aware scheduling algorithms, recurrent neural networks) to establish intervention time constraints and prioritize medical needs accordingly.
(iii) Resource and personnel matching module: This module employs optimization algorithms and knowledge-based reasoning to align required equipment and healthcare professional profiles to intervention demands, generating resource requirement vectors.
(iv) Hospital category selection engine: This module combines the above outputs with hospital capability data via multi-criteria decision analysis or machine learning classification models (e.g., multi-class SVM, Random Forest classifiers). This engine evaluates which hospital categories fully meet or exceed the intervention needs, timing constraints, and resource/personnel availability.
(v) Continuous learning and validation loop: This module incorporates feedback from actual treatment outcomes, hospital performance metrics, and new clinical guidelines to iteratively update intervention mapping, timing models, and hospital capability assessments.

The outputs of this machine-learning model include:
(i) an explicit determination of medical interventions necessary for the patient, listing surgery, treatments, therapies, or combinations required.
(ii) the recommended timeframes within which each intervention should ideally be administered to maximize clinical effectiveness.
(iii) identification of the equipment and healthcare professionals required to perform each intervention.
(iv) a prioritized selection of one or more categories of hospitals that possess the capability, equipment, personnel, and timing capacity to deliver the prescribed interventions effectively.
(v) justifications or confidence scores supporting the selection, facilitating clinical decision-making and emergency dispatch planning.

The description is continued below with respect to the manner in which ERS management system 140 selects the category of ambulances.

### Selection of the Category of Ambulances

ERS management system 140 provides inputs related to the selection of category of ambulance based on patient-specific medical requirements, to a third machine-learning model. The input modalities include:
(i) Determined medical interventions and timing: This includes outputs from the second machine-learning model, including, the medical interventions and their required timeframes for the patient (e.g., surgery, treatment, therapy, combinations thereof, and the urgency of each), derived from the patient health assessment and intervention determination modules.
(ii) Patient physical profile: This includes profile of the patient, including age, physique (weight, height, body mass index (BMI)), and other parameters influencing the type of equipment and medical facilities needed onboard.
(iii) On-board medical facilities database: This includes a structured repository describing possible ambulance categories and their corresponding in-vehicle medical facilities, such as advanced life support (ALS) units, cardiac monitoring, ventilators, defibrillators, trauma care kits, infusion pumps, and other specialized medical installations.
(iv) On-board equipment inventory: This includes data detailing medical devices and portable equipment that can be carried or integrated within ambulances, including consumables and apparatus like oxygen tanks, suction devices, medication kits, immobilization tools, and monitoring instruments.
(v) Healthcare professional profiles: This includes information regarding the clinical qualifications, specialties, certifications, and availability of healthcare personnel who can staff ambulances, including paramedics, emergency medical technicians (EMTs), nurses, physicians, respiratory therapists, and specialists.
(vi) Ambulance categorization database: A classification scheme categorizing ambulance types based on their medical facility capabilities, equipment configurations, healthcare staff complement, and operational readiness to deliver specified emergency care levels.

Features are extracted from the above inputs as follows:
(i) Medical facility requirements: This includes mapping patient medical intervention types and urgency (timing) to required on-board medical facilities. For example, a patient needing acute respiratory therapy within a critical time window may require an ambulance equipped with ventilators and oxygen supply systems. This also includes encoding combinations of medical intervention needs to determine composite facility requirements.
(ii) Equipment necessity features: This includes using patient physical profile parameters and medical facility requirements to deduce the specific equipment set needed onboard (e.g., size-appropriate immobilization devices for patient weight, specific medication availability). This also includes including dynamic considerations such as anticipated intervention complexity or duration influencing equipment load.
(iii) Healthcare professional staffing features: This includes matching required medical facilities and equipment with the qualifications and certifications needed among ambulance staff to operate them safely and effectively; and incorporating availability and experience level of personnel to reflect operational reality.
(iv) Ambulance capability encoding: This includes representing ambulance categories as multidimensional vectors indicating what medical facilities they support, equipment onboard, and staff levels typically assigned; and combining these with logistical parameters like response time capabilities, vehicle range, and jurisdictional constraints.

The extracted features are integrated into a cohesive machine learning pipeline containing:
(i) Medical facility requirement module: This module employs predictive or rule-based models (e.g., gradient boosting, feed-forward neural networks) trained on historical patient intervention and ambulance care data to infer the necessary on-board medical facilities for the ambulance given patient intervention timing and condition. Training data includes prior ambulance dispatch records linked with patient outcomes and intervention types.
(ii) Equipment determination module: This module analyses patient physical profiles and determined medical facilities to predict optimal on-board equipment configurations. This module may use supervised learning methods, incorporating multi-modal data vectors and expert system heuristics to generate equipment requirement sets.
(iii) Healthcare professional determination module: This module uses classification or recommendation algorithms to identify the appropriate healthcare professional mix for the ambulance crew, based on required medical facilities, equipment to be operated, and patient complexity indicators. This includes ensuring that staff possess necessary certifications and skills for interventions anticipated.
(iv) Ambulance category selection engine: This module engine outputs from the above modules with ambulance category profiles in a multi-criteria decision-making model or machine learning classifier (e.g., multi-class SVM, Random Forest, or neural network). This engine matches patient-specific on-board medical facility, equipment, and staffing needs with ambulance capabilities to select one or more ambulance categories that optimally fulfil those requirements.
(v) Continuous feedback and adaptation loop: This incorporates outcome metrics such as patient treatment success, response times, equipment usage logs, and staff performance reviews to iteratively refine model predictions and hospital/ambulance categorization criteria over time.

The outputs of this machine-learning model include:
(i) a detailed specification of required on-board medical facilities for the desired ambulance, aligned with patient interventions and timing needs.
(ii) a recommended list of on-board equipment tailored to the patient's physical profile and medical condition to enable effective emergency care during transport.
(iii) identification of necessary healthcare professionals to staff the ambulance, ensuring proper skills and certification levels relative to equipment and intervention complexity.
(iv) the selected ambulance category or categories from a broader set, each meeting or exceeding the above criteria, ready for dispatch with appropriate medical support capabilities.
(v) supporting data such as confidence scores and justifications for selections, assisting emergency response coordinators in decision-making.

The description is continued below with respect to the manner in which ERS management system 140 determines/selects one or more ambulances from the selected category of ambulances.

### Determining One or More Ambulance From the Selected Category of Ambulances

In an example implementation ERS management system 140 determines the one or more of the ambulances from the selected category of ambulances using a fourth machine-learning model. The input modalities of this machine-learning model include:
(i) Real-time GPS coordinates and operational status reports from all ambulances within the selected category (e.g., ALS units). The coordinates are updated for pre-defined time intervals (e.g., every 5 seconds).
(ii) Digital road network data containing detailed maps of the area including road segments, intersections, speed limits, and possible routing alternatives in geospatial formats such as shape files or graph data structures.
(iii) Weather data gathered from meteorological services providing forecasts and current conditions (temperature, precipitation, visibility) at specific geolocations and timestamps corresponding to ambulance routes.
(iv) Dynamic traffic data feeds indicating current congestion levels, estimated waiting times at traffic signals, and incident reports received from municipal and third-party traffic monitoring systems.
(v) Data feeds on scheduled or unscheduled public events and VIP movements that can potentially restrict or slow traffic on certain road segments. The data feeds are timestamped and geotagged.
(vi) Historical data repositories storing records of ambulance routing logs, travel times under varying conditions, and patient condition severity metrics.

Each input modality is ingested in a digital format appropriate for subsequent processing, with timestamps synchronized to allow correlation across sources.

The raw inputs are transformed into actionable features by:
(i) Encoding ambulance locations into spatial coordinate vectors and associating each ambulance with metadata indicating category, availability, and current deployment status.
(ii) Representing roads and routes as nodes and edges in graph structures, enriched with attributes such as estimated traversal times, traffic signal delays, and road conditions.
(iii) Temporal encoding of weather forecasts and traffic condition trends into time series features aligned with predicted ambulance travel windows.
(iv) Event and VIP movement information are mapped to spatial-temporal buffers affecting traffic flow probabilities along relevant routes.
(v) Patient medical severity scores and other triage features normalized and embedded as scalar inputs for assignment prioritization.
(v) Applying data normalization, smoothing, and windowing techniques to minimize noise and handle missing values.

The above steps produce multidimensional feature vectors that characterize the dispatch environment at fine spatial and temporal granularity.

The machine-learning model integrates the above features through specialized machine learning components:

A Demand Forecasting Engine utilizing Long Short-Term Memory (LSTM) networks combined with gradient boosting machines forecasts spatial-temporal emergency call intensity hotspots for strategic ambulance pre-positioning. Training is performed on historical and real-time labeled data, using a supervised learning approach optimizing prediction accuracy metrics.

An Ambulance Assignment Module applies classifiers such as Support Vector Machines (SVM) and Decision Trees trained on multimodal features containing patient severity, ambulance proximity, availability status, and predicted demand. This module outputs a ranked list of one or more ambulances within the selected category that are deemed optimal for dispatch. Training is based on historical dispatch outcomes and operational criteria reflecting response priorities.

A Route Optimization Engine implements a hybrid deep neural network architecture combining Convolutional Neural Networks (CNN) and Graph Neural Networks (GNN) to extract spatial features from road topology data, and sequential models like LSTM or Gated Recurrent Units (GRU) to model flow and delays due to traffic, weather, and events. This engine dynamically computes optimal driving routes from the selected ambulance(s)' locations to the patient's site, minimizing estimated travel and delay times.

A Continuous Feedback Loop collects outcome metrics such as actual response times, route deviations, and patient outcomes, incorporating them into periodic retraining and fine-tuning of the models. This adaptive mechanism enables ongoing performance improvements and model robustness to changing urban conditions.

The outputs of the machine-learning model include a specific set of one or more ambulances selected from the selected category of ambulances, ranked and justified based on multi-factorial predictive analysis.

The description is continued below with respect to the manner in which ERS management system 140 determines/selects one or more hospitals from the selected category of hospitals.

### Determining One or More Hospitals From the Selected Category of Hospitals

In an example implementation ERS management system 140 determines the one or more of the ambulances from the selected category of ambulances using a fifth machine-learning model. The input modalities of this machine-learning model include:
(i) Real-time and static location data of hospitals belonging to the selected category (e.g., trauma centers, specialized care units). Hospital locations are captured as geographic coordinates (latitude/longitude) along with metadata such as specialization, capacity, operational status, and current patient load.
(ii) Patient location data: This data includes current geospatial coordinates of the patient requiring hospital services, obtained via GPS or emergency dispatch information.
(iii) Road network information: This data includes digital maps describing available routes from the patient's location to each hospital location, including road segments, intersections, one-way streets, and alternative path options.
(iv) Dynamic environmental data: This data includes weather forecasts and real-time conditions (e.g., rain, snow, fog) along each possible route, affecting travel safety and speed; traffic conditions including current congestion levels, accidents, or roadworks., and estimated wait times at traffic signals along routes.
(v) Special event data: This data includes scheduled or ongoing public events and VIP movements that may impact traffic flow or accessibility on routes between the patient and hospitals.
(vi) Historical data: This data includes past travel times, route usage statistics, hospital admissions, and emergency case outcomes associated with different hospitals, routes, and environmental conditions.

The above inputs are digitally timestamped and geotagged to allow spatio-temporal correlation and integrated processing.

From the raw multimodal inputs, the system extracts features encapsulating spatial, temporal, and contextual aspects critical to hospital selection, as below:
(i) Spatial features: These features include patient and hospital geographic coordinates mapped in a spatial reference system, and road network topology represented as graph nodes and weighted edges reflecting distances and potential travel costs.
(ii)Temporal features: These features include time-varying traffic congestion patterns along candidate routes, weather condition time series corresponding to the expected travel period, and traffic signal timing and wait time fluctuations.
(iii) Contextual features: These features include hospital-specific metadata such as treatment capabilities, capacity, and operational status (e.g., occupied beds, ICU availability), and public event and VIP movement impacts encoded as probabilistic traffic disruption indicators on relevant route segments.
(v) Statistical/historical features: These features include historical travel time distributions along routes under varying conditions, and prior hospital performance data linked to patient outcomes.

Feature engineering techniques, including normalization, embeddings, and temporal windowing, can be performed to refine the data for input into machine learning modules.

The system integrates these features within a modular machine learning architecture arranged as follows:
(i) Hospital demand and suitability forecasting module: This module employs spatio-temporal predictive models such as Long Short-Term Memory (LSTM) networks and gradient boosting machines to analyze historical and real-time data, estimating hospital demand patterns and suitability indices. This facilitates anticipating capacity constraints and selecting hospitals with optimal resource availability.
(ii) Hospital selection module: This module utilizes classification algorithms, for example, Support Vector Machines (SVM), Random Forests, or Decision Trees, trained on composite feature vectors including patient location, hospital metadata, distance, travel time estimates, and environmental conditions. This module outputs a prioritized set of one or more hospitals within the selected category considered optimal candidates for receiving the patient.
(iii) Route evaluation and optimization engine: This module applies hybrid deep learning architectures merging Convolutional Neural Networks (CNN) and Graph Neural Networks (GNN) to model the spatial complexity of possible routes, combined with recurrent layers (LSTM or Gated Recurrent Units, GRU) to capture temporal fluctuations in traffic, signal delays, weather impact, and events. This engine dynamically estimates travel times and reliability scores across routes linking the patient with candidate hospitals.
(iv) Adaptive feedback loop can be used to continuously collect operational performance data including actual hospital arrival times, route adherence, hospital capacity changes, and patient outcomes. This feedback informs periodic retraining and fine-tuning of all model components, enhancing prediction accuracy and system responsiveness to evolving conditions. This model enables the real-time fusion of multimodal data to support informed hospital selection and routing decisions under uncertain, dynamic urban environments.

The outputs of the machine-learning module include the selection of one or more hospitals from the selected category of hospitals, ranked and justified based on integrated predictive analytics considering travel efficiency, hospital capacity, and treatment suitability. The description is continued below with respect to example implementations of the aspects of the present disclosure.

### Example Implementations

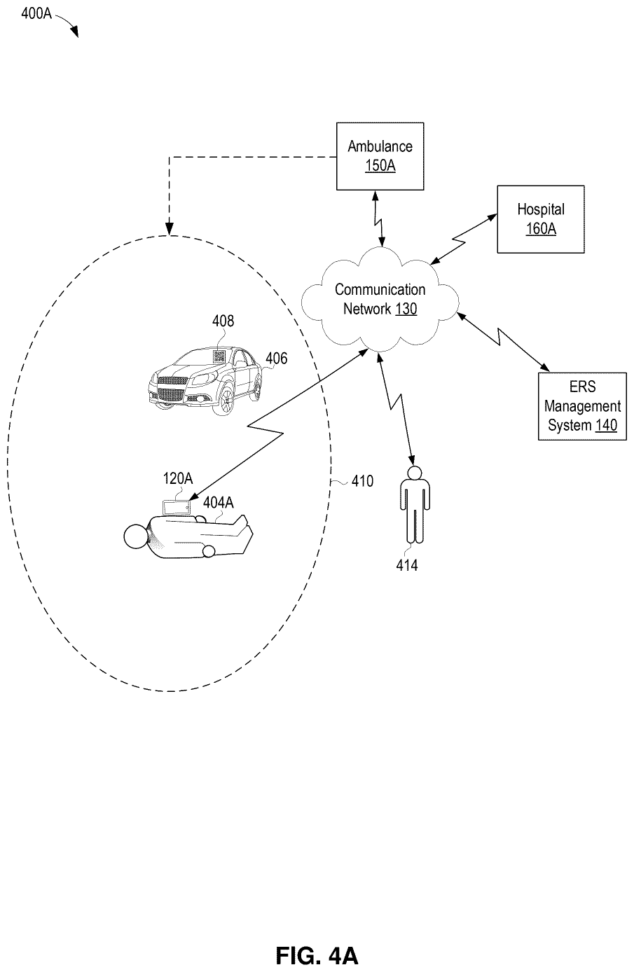

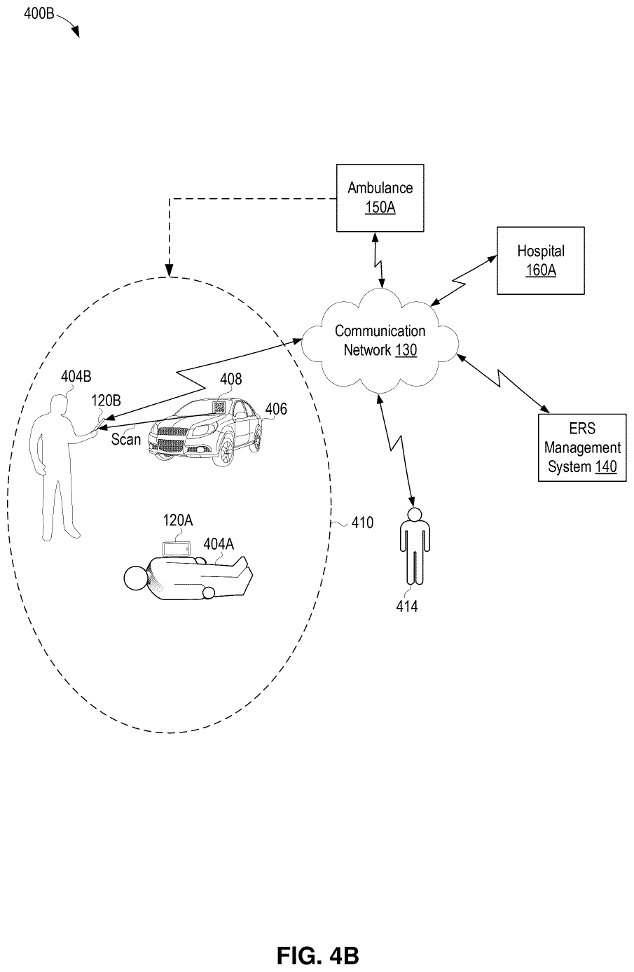

FIG. 4A and FIG. 4B depict example implementation environments of the aspects of the present disclosure. FIG. 4A depicts example implementation environment 400A where the emergency information is sent by the patient himself, and FIG. 4B depicts example implementation environment 400B where the emergency information is sent by a bystander.

Example implementation environment 400A is shown containing patient 404A with user device 120A (a mobile phone), car 406 with QR code 408, communication network 130, ERS management system 140, ambulance 150A, hospital 160A, and emergency contact 414. Patient 404A, user device 120A and car 406 (with QR code 408) are all shown at location 410. Environment 400A shows a representative set of components for illustration only. The description continues below, assuming that patient 404A sustained injuries in an accident.

Patient 404A sends a unique identifier to ERS management system 140 from a mobile application running on user device 120A. ERS management system 140 receives the unique identifier and sends a prompt to user device 120A, asking patient 404A to confirm that the subscriber (patient 404A) is in an emergency situation at location 410 (i.e., the location from which the unique identifier was sent). Patient 404A receives the prompt in the mobile application and responds with confirmation, which ERS management system 140 receives from user device 120A. ERS management system 140 then determines the location of user device 120A based on location data obtained from the mobile application. Next, ERS management system 140 prompts patient 404A to send emergency information related to the subscriber. Patient 404A sends emergency information including a description of his injuries and a video depicting his current condition, via user device 120A, to ERS management system 140.

ERS management system 140 retrieves the profile (containing physique, age, and gender) and emergency contact 414 of patient 404A using the received unique identifier from its database. ERS management system 140 assesses the health condition of patient 404A based on the emergency information and profile received, using the first machine learning model. ERS management system 140 then selects a category of hospitals based on the health assessment and the profile using a second machine learning model, and selects a category of ambulances based on the health assessment and the profile using the third machine learning model. Subsequently, ERS management system 140 selects ambulance 150A using the fourth machine learning model, and selects hospital 160A using the fifth machine learning model.

ERS management system 140 simultaneously sends a first alert, including the location of patient 404A and details of hospital 160A, to ambulance 150A; a second alert, including the patient's health condition, to hospital 160A; and a communication to emergency contact 414. The communication to emergency contact 414 includes the real-time location of patient 404A, real-time location of ambulance 150A, location of hospital 160A, and contact details for both ambulance 150A and hospital 160A. ERS management system 140 guides the ambulance to the patient and then to the hospital using another machine learning model. Additionally, it facilitates automated registration of patient 404A with hospital 160A using the unique identifier and a medical insurance account of patient 404A retrieved from the subscriber's record in the database.

Turning now to example implementation environment 400B, example implementation environment 400B is shown containing patient 404A with user device 120A, bystander 404B with user device 120B, car 406 with QR code 408, communication network 130, ERS management system 140, ambulance 150A, hospital 160A, and emergency contact 414. Patient 404A, user device 120A, patient 404B, user device 120B, and car 406 (with QR code 408) are all shown at location 410. Environment 400B shows a representative set of components for illustration only.

The operation of example implementation environment 400B is similar to that of example implementation environment 400A, except that in environment 400B, bystander 404B scans the QR code 408 using user device 120B and sends the unique identifier to ERS management system 140, and subsequent communications happen between user device 120B and ERS management system 140.

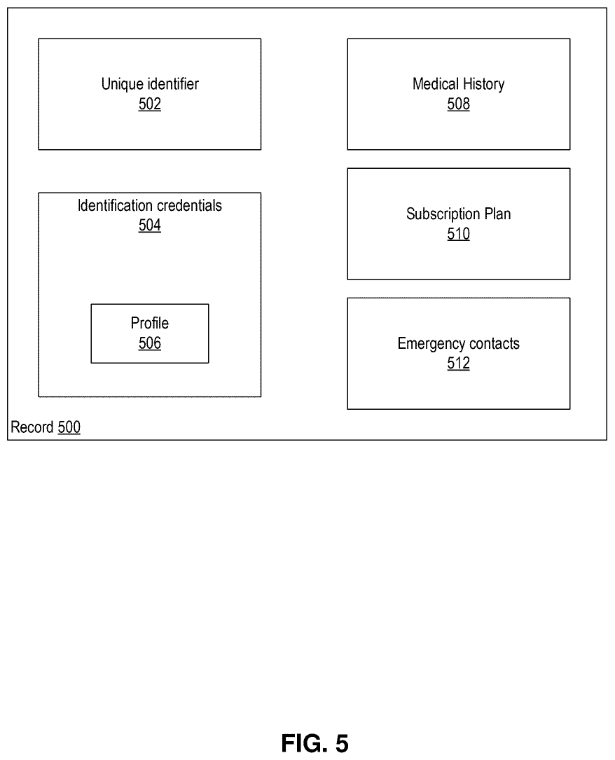

FIG. 5 illustrates example record 500, corresponding to a subscriber, stored in the database associated with ERS management system 140. FIG. 5 is shown containing unique identifier 502, identification credentials 504, medical history 508, subscription plan 510, and emergency contacts 512. Identification credentials 504 is further shown containing profile 506.

Unique identifier 502 is an identifier that uniquely identifies corresponding subscriber. Unique identifier 502 is assigned to every subscriber upon registration with ERS management system 140. Unique identifier can be an alphanumeric code, or a numeric code, or a QR code. In an example implementation, unique identifier is a QR code. The QR code can be in a digital form or in a tangible form printed on a tangible material. Examples of the tangible material include, but are not limited to, a card, a paper, a sticker and the like. The tangible medium can be attachable to an object or a person.

Identification credentials 504 of the subscriber include details such as name of the subscriber, email ID of the subscriber, date of birth of the subscriber, contact number of the subscriber, social security number of the subscriber, a driving license number, a medical ID, a medical insurance ID (medical insurance account), and digital wallet details of the subscriber (a digital wallet contains funds for emergency services), and profile 506 of the subscriber. Profile 506 includes details such as age of the subscriber, physique of the subscriber, and gender of the subscriber.

Medical history 508 contains the past health information of the subscriber. Subscription plan 510 contains subscription details of the subscriber such as type of subscription. In an example, ERS management system 140 offers various subscription plans tailored to cover various emergency expenses. For example, a first subscription plan offers a preloaded credit of INR 50,000 (Fifty thousand rupees) and can be used to cover hospitalization and emergency care expenses for a duration of 24 hours; and a second subscription plan offers a preloaded credit of INR 1,00,000 (One lakh rupees) and provides coverage for hospitalization expenses for up to 48 hours, minor surgeries such as stitches, and medicines up to INR 10,000.

Emergency contacts 512 of the subscriber include the contact numbers of the designated persons who are to be notified if the subscriber is in an emergency situation. In addition, emergency contacts 512 may also contain the names and the addresses of the designated persons.

In an example implementation, record 500 can be accessed with the QR code as the primary key.

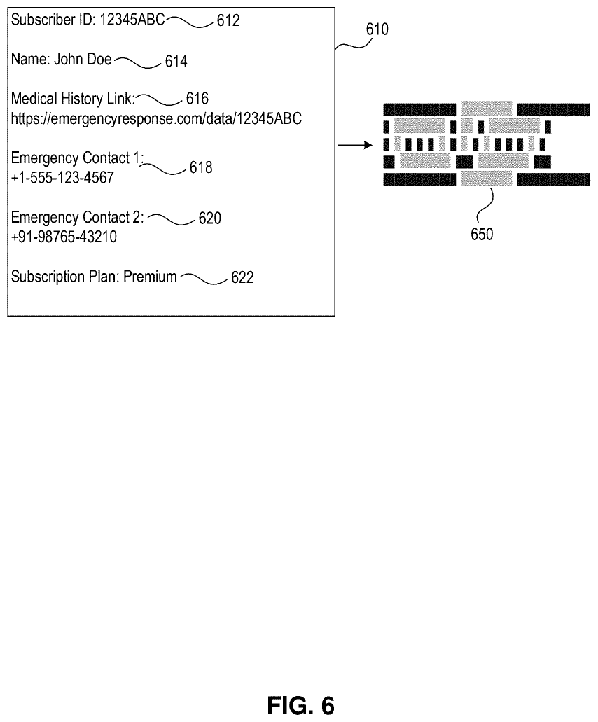

FIG. 6 depicts details 610 of the subscriber linked to QR code 650. Details 610 include subscriber ID 612 with 12345ABC as corresponding value, name 614 with “John Doe” as corresponding value, medical history 616 with weblink “https://emergencyresponse.com/data/12345ABC” as value, emergency contact1 618 with+1-555-123-4567 as corresponding value, emergency contact 2 620 with +91-98765-43210, and subscription plan 622 with “Premium” as the corresponding value.

Details 610 are linked to QR code 650 at the time of registration, and QR code 650 is stored in the database of ERS management system 140.

When the QR code 650 is scanned using user device 120 (for example, a smartphone), emergency response system 140 is directed to the subscriber's record in the database. Alternatively, the digital code could simply be a unique alphanumeric code (e.g., QR-00123XYZ), which, when entered into an emergency response application, ERS management system 140 retrieves the subscriber-specific details stored in the database.

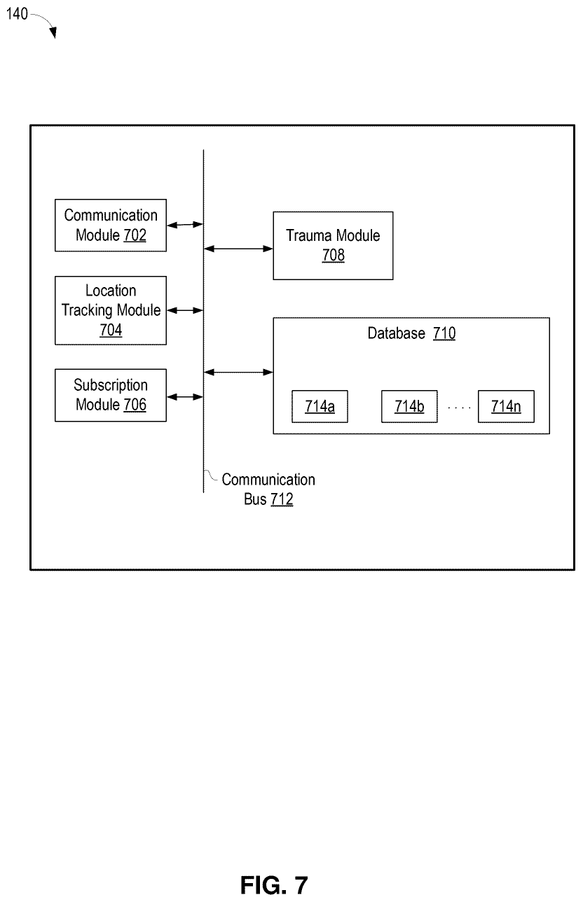

FIG. 7 depicts a block diagram of ERS management system 140, designed for managing emergency response services, according to an example embodiment. ERS management system 140 is shown containing communication module 702, location tracking module 704, subscription module 706, trauma module 708, database 710, and communication bus 712. These modules are communicatively coupled via a communication bus 712. Database 710 stores various information in data blocks 714a-714n, associated with the subscribers. ERS management system 140 is accessible from user device 120 of a subscriber or any third party by scanning a digital code/QR code associated with the subscriber. The functions of each module are explained hereinbelow.

Subscription module 706 is programmed to register subscribers with ERS management system 140 by assigning and activating a unique digital code for each subscriber. The unique digital code is provided to each subscriber upon enrolment/registering by purchasing the code. In one example, the digital code can be a QR code.

To activate a QR code, a subscriber accesses ERS management system 140 by logging into an online application (web or mobile) and either entering or scanning the QR code within the application. The subscriber then inputs a one-time password (OTP) and a mobile number for user authentication. Upon submission, subscription module 706 receives the OTP and mobile number and activates the QR code by linking it to the subscriber. This process ensures that the QR code is securely assigned and ready for use within ERS management system 140. After activating the QR code, the application requests the subscriber's identification credentials to complete registration. Examples of identification credentials include, but are not limited to, name, email ID, date of birth, social security number, driver's license number, medical ID, medical insurance ID, and phone number. Subscription module 706 receives these credentials along with the QR code, links them together, and stores the information in the database 710.

Subscription module 706 further enables the subscriber to select a subscription plan for availing the emergency response services using the QR code. The subscription plan is preloaded with a predetermined amount of money to cover one or more emergency expenses, hospitalization, medical treatment costs, and death cover.

In one example, ERS management system 140 offers multiple subscription plans tailored to cover specific emergency expenses. Subscribers select a plan when registering their QR code and pay accordingly. The chosen subscription plan is stored in the database 710 and linked to the QR code, ensuring seamless access to emergency services.

Further, subscription module 706 receives various information associated with each subscriber. This information includes medical history, medical insurance plans, and one or more emergency contacts. Typically, this information is provided by the subscriber via the online application and transmitted to subscription module 706. Subscription module 106 links the subscriber information, identification credentials, medical history, emergency contacts, and subscription plan to the QR code and stores these details in the database 710.

Once the registration of the subscriber and activation of the QR code associated with the subscriber are complete, the ERS management system 140 is ready to provide the necessary emergency response services. In case the subscriber meets with an accident or emergency and needs help, the subscriber can activate the emergency response service by triggering the QR code via an online or client application available on user device 120 (mobile or portable device) capable of communicating with ERS management system 140. The QR code can be triggered by entering the code into the application or by scanning it. Upon scanning, a digital copy of the QR code along with a request for emergency response services is communicated to ERS management system 140.

Communication module 702 receives the digital copy of the QR code from the user device and identifies the subscriber linked to the QR code by querying database 710. Communication module 702 then prompts the user of the device to confirm the occurrence of an emergency involving the subscriber at the location from which the QR code was transmitted. In one embodiment, communication module 702 may also send a photograph of the subscriber, retrieved from database 710, to the user device to assist in verification and requests confirmation of the emergency. If the user confirms that an emergency has occurred at the location and involves the subscriber after viewing the photograph, communication module 702 retrieves the location of the user device. If the user indicates no emergency—such as when the QR code was scanned out of curiosity or for any non-emergency reason—the communication module 702 disregards the request.

Upon receiving confirmation of an emergency, communication module 702 sends an emergency message regarding the subscriber and the incident to one or more emergency contacts linked to the QR code. Contact details of these emergency contacts are retrieved from database 710. Communication module 702 also prompts the user to capture one or more real-time photographs and videos (hereinafter referred to interchangeably as media) of the subscriber to assess the current medical condition and its severity. The media captured is shared with trauma module 708 for analysis.

Trauma module 708 is configured to identify the current medical condition and severity based on the real-time media received from communication module 702 and the subscriber's medical history retrieved from database 710. The identified medical condition may include the type of injury, severity, and any medical abnormalities. Trauma module 708 employs a machine learning model trained on numerous photographs and videos of various individuals, enabling it to accurately determine the type and severity of medical conditions. The model also incorporates the subscriber's medical history during analysis, achieving an identification accuracy of up to 90%.

Based on the assessed health condition, trauma module 708 recommends an ambulance category from multiple available categories. By selecting the most suitable ambulance category tailored to the subscriber's medical condition, trauma module 708 optimizes emergency response and facilitates timely medical care.

Location tracking module 704 selects an ambulance within the recommended category. It directs the ambulance to the subscriber's location by transmitting real-time GPS signals that pinpoint the exact geographic coordinates. The location tracking module 704 also recommends one or more hospitals. ERS management system 140 maintains a dynamic, comprehensive list of regional hospitals along with their available facilities and regularly updates this list through real-time data sources such as news feeds.

When an emergency occurs, location tracking module 704 efficiently searches this database to identify hospitals that have the necessary infrastructure and specialist staff to treat the condition and are in close proximity to the subscriber. Based on this information, ERS management system 140 recommends the hospital best suited to provide rapid and effective medical intervention.

For example, if a subscriber suffers a severe eye injury requiring specialized treatment with equipment such as a vitrectomy machine for retinal surgery, which is typically unavailable in general hospitals, Trauma module 708 and location tracking module 704 collaboratively recommend a hospital that has both the necessary equipment and an ophthalmic surgeon on call. This targeted recommendation significantly improves the chance of a successful recovery.

In such eye care emergencies, prompt deployment of suitable medical resources is critical to preserving vision. ERS management system 140, aided by location tracking module 704 and its hospital recommendations, reduces patient response times to approximately 20 minutes—far shorter than the hour or more often experienced with existing solutions—thereby enhancing treatment outcomes.

Similarly, for injuries necessitating specialized equipment such as an Optical Coherence Tomography (OCT) device or a Laser Photocoagulator for retinal repair, the ERS management system 140 quickly identifies and recommends hospitals equipped with these technologies and staffed with ophthalmic surgeons. This swift direction to the right facility safeguards the patient from further damage and optimizes prognosis and recovery.

Communication module 702 periodically sends status updates regarding the subscriber to the emergency contacts. These updates include the identified health condition, ambulance contact details, recommended hospitals, and real-time locations of both the ambulance and subscriber. Communication module 702 also receives feedback from the emergency contacts and can facilitate communication between them and the ERS management system's emergency response team.

Upon arrival at the hospital, trauma module 708 facilitates automated registration of the subscriber as a patient using the QR code and provisions cashless emergency care, hospitalization, medical treatment, or any combination thereof as per the subscription plan linked to the QR code. This process ensures seamless transport and rapid delivery of optimal medical care to the subscriber.

Thus, ERS management system 140 of the present disclosure efficiently assesses patient health condition using injury descriptions, health issues, and visual media; determines patient location; and selects appropriate hospital and ambulance categories. ERS management system 140 also enhances assessment accuracy by incorporating patient profile (physical, gender, medical history) and disaster-related information like epidemics and local hazards. Also, ERS management system 140 of the present disclosure enables precise hospital category selection based on required medical interventions (surgery, treatment, therapy), timing, needed equipment, and healthcare professionals; and precise ambulance category selection by specifying on-board medical facilities, equipment, and healthcare professionals tailored to the patient's needs and profile. ERS management system 140 of the present disclosure optimizes ambulance dispatch by considering ambulance locations, routes, weather, traffic, wait times, VIP movements, and public events to reduce response time. ERS management system 140 selects hospitals based on location, routes, weather, traffic, wait times, VIP movements, and public events to ensure timely and efficient patient transport. ERS management system 140 provides dynamic selection of hospital and ambulance categories based on varying patient health conditions and profiles for personalized emergency response, and supports storage of subscriber records with unique identifiers and detailed profiles, improving response personalization and efficiency. ERS management system 140 of the present disclosure facilitates patient identification and emergency confirmation via user devices, enabling precise location tracking and profile retrieval to inform response. ERS management system 140 integrates emergency contacts, medical insurance, and digital wallets for streamlined communication, automated hospital registration, and financial facilitation, improving overall service coordination. ERS management system 140 utilizes a multimodal machine-learning model combining emergency information, patient profile, and disaster data for accurate health condition assessment and informed decision-making.

The technical effect and solution provided by the present disclosure are manifold. The present disclosure offers a technologically advanced and efficient approach to managing emergency response services by integrating real-time data assessment, automated decision-making, and intelligent communication. One key advantage is the system's ability to rapidly evaluate the patient's present health condition using descriptive data and visual media, enabling the selection and dispatch of the most suitable category of ambulance and hospital. This leads to faster and more context-aware emergency response, which is critical in life-threatening situations.

Another significant benefit lies in the personalized decision-making enabled by the system. By incorporating patient-specific profile information such as physique, age, and medical history, the system ensures that emergency services are not only quick but also tailored to the unique medical needs of the patient. This increases the likelihood of better medical outcomes by ensuring the patient is routed to a facility equipped to handle their specific condition. The system also streamlines communication during emergencies by automatically notifying the patient's emergency contacts. These notifications may include real-time updates such as the patient's location, ambulance location, and hospital information, thereby reducing panic and improving coordination among family members or caregivers.

Aspects of the present disclosure also enable a highly personalized, context-aware, and optimized emergency response system that improves patient care and resource allocation.

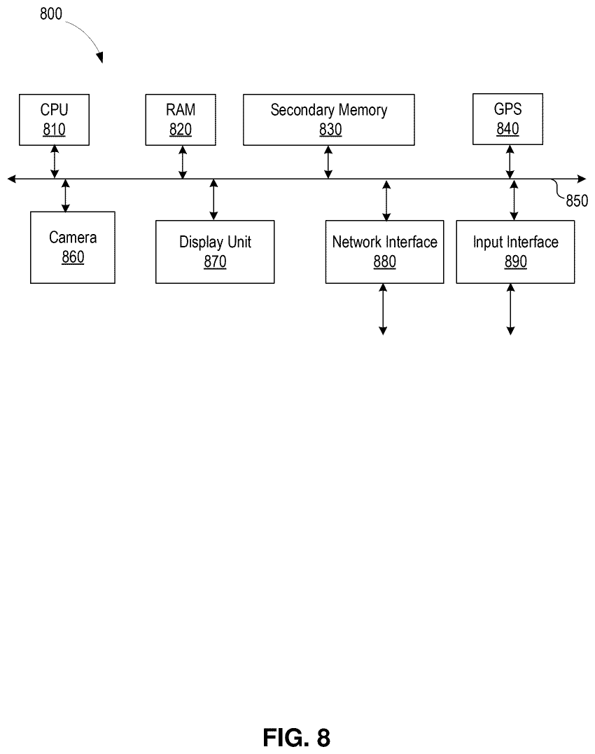

Description is continued below with respect to the description of FIG. 8.

FIG. 8 is a block diagram illustrating the details of digital processing system 800 in which various aspects of the present disclosure are operative by execution of appropriate executable modules. Digital processing system 800 corresponds to user device 120 and ERS management system 140, and also any communication devices in ambulances 150 and hospitals 160.

Digital processing system 800 may contain one or more processors such as a central processing unit (CPU) 810, random access memory (RAM) 820, secondary memory 830, GPS 840, camera 860, display unit 870, network interface 880, and input interface 890. All the components may communicate with each other over communication path 850. The components of FIG. 8 are described below in further detail. CPU 810 executes instructions stored in RAM 820 to provide several features of the present disclosure. CPU 810 may contain multiple processing units or a single processing unit. RAM 820 may receive instructions from secondary memory 830, via communication path 850. RAM 820 may contain software instructions and application programs, facilitates a (common) run time environment for execution of user programs.

Display unit 870 displays based on data/instructions received from CPU 810. Display unit 870 contains a display screen to display images. Network interface 880 facilitates connectivity to a network. Input interface 890 may correspond to a keyboard and a pointing device (e.g., touch-pad, mouse) that may be used to provide appropriate inputs.

Secondary memory 830 stores the data and software instructions for implementing the flowchart of FIGS. 2 and 3, which enable digital processing system 800 to provide several features in accordance with the present disclosure. The code/instructions stored in secondary memory 830 either may be copied to RAM 820 prior to execution by CPU 810, or may be directly executed by CPU 810.

CPU 810 may retrieve the software instructions, and execute the instructions to provide various features of the present disclosure described above.

Storage media is distinct from but may be used in conjunction with transmission media. Transmission media participates in transferring information between storage media. For example, transmission media includes coaxial cables, copper wire and fiber optics, including the wires that comprise bus 850. Transmission media can also take the form of acoustic or light waves, such as those generated during radio-wave and infra-red data communications.

GPS 840 is a satellite-based navigation system that enables digital processing system 800 to determine its precise geographic location (latitude, longitude, and altitude) anywhere on or near the Earth. The GPS receiver of digital processing system 800 communicates with a constellation of satellites that continuously transmit signals containing their positions and exact time. By calculating the time delays of these signals from multiple satellites through a process called trilateration, the GPS receiver accurately computes the device's location and can also provide speed and direction information. This technology facilitates navigation, mapping, location-based services, and timing synchronization in mobile devices.

Camera 860 is an image-capturing sensor system that allows digital processing system 800 to capture photos and record videos. It typically consists of a lens, image sensor (such as a CMOS sensor), and associated electronics to convert light into digital signals. Camera 860 interfaces with processor 810 to transmit image data and receive control commands, enabling features like autofocus, exposure control, and image processing. Camera 860 supports applications such as photography, video calling, augmented reality, and scanning.

It will be understood by those within the art that, in general, terms used herein, are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

While only certain features of several embodiments have been illustrated, and described herein, many modifications and changes will occur to those skilled in the art. It is, therefore, to be understood that the appended claims are intended to cover all such modifications and changes as fall within the true spirit of inventive concepts.

The aforementioned description is merely illustrative in nature and is in no way intended to limit the disclosure, its application, or uses. The broad teachings of the disclosure may be implemented in a variety of forms. Therefore, while this disclosure includes particular examples, the true scope of the disclosure should not be so limited since other modifications will become apparent upon a study of the drawings, the specification. It should be understood, that one or more steps within a method may be executed in different order (or concurrently) without altering the principles of the present disclosure. Further, although each of the example embodiments is described above as having certain features, any one or more of those features described with respect to any example embodiment of the disclosure may be implemented in and/or combined with features of any of the other embodiments, even if that combination is not explicitly described. In other words, the described example embodiments are not mutually exclusive, and permutations of one or more example embodiments with one another remain within the scope of this disclosure.

The example embodiment or each example embodiment should not be understood as a limiting/restrictive of inventive concepts. Rather, numerous variations and modifications are possible in the context of the present disclosure, in particular those variants and combinations which may be inferred by the person skilled in the art with regard to achieving the object for example by combination or modification of individual features or elements or method steps that are described in connection with the general or specific part of the description and/or the drawings, and, by way of combinable features, lead to a new subject matter or to new method steps or sequences of method steps, including insofar as they concern production, testing and operating methods. Further, elements and/or features of different example embodiments may be combined with each other and/or substituted for each other within the scope of this disclosure.

Still further, any one of the above-described and other examples features of example embodiments may be embodied in the form of an apparatus, method, system, computer program, tangible computer readable medium and tangible computer program product. For example, the aforementioned methods may be embodied in the form of a system or device, including, but not limited to, any of the structures for performing the methodology illustrated in the drawings. In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Further, at least one example embodiment relates to a non-transitory computer-readable storage medium comprising electronically readable control information (e.g., computer-readable instructions) stored thereon, configured such that when the storage medium is used in a controller of a magnetic resonance device, at least one example embodiment of the method is carried out.

Even further, any of the aforementioned methods may be embodied in the form of a program. The program may be stored on a non-transitory computer readable medium, such that when run on a computer device (e.g., a processor), cause the computer-device to perform any one of the aforementioned methods. Thus, the non-transitory, tangible computer readable medium is adapted to store information and is adapted to interact with a data processing facility or computer device to execute the program of any of the above-mentioned embodiments and/or to perform the method of any of the above-mentioned embodiments.

The computer readable medium or storage medium may be a built-in medium installed inside a computer device's main body or a removable medium arranged so that it may be separated from the computer device's main body. The term computer-readable medium, as used herein, does not encompass transitory electrical or electromagnetic signals propagating through a medium (such as on a carrier wave), the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices), volatile memory devices (including, for example static random access memory devices or a dynamic random access memory devices), magnetic storage media (including, for example an analog or digital magnetic tape or a hard disk drive), and optical storage media (including, for example a CD, a DVD, or a Blu-ray Disc). Examples of the media with a built-in rewriteable non-volatile memory, include but are not limited to memory cards, and media with a built-in ROM, including but not limited to ROM cassettes, etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave), the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices), volatile memory devices (including, for example static random access memory devices or a dynamic random access memory devices), magnetic storage media (including, for example an analog or digital magnetic tape or a hard disk drive), and optical storage media (including, for example a CD, a DVD, or a Blu-ray Disc).

Examples of the media with a built-in rewriteable non-volatile memory, include, but are not limited to memory cards, and media with a built-in ROM, including but not limited to ROM cassettes, etc. Furthermore, various information regarding stored images, for example, property information, may be stored in any other form, or it may be provided in other ways.

The apparatuses and methods described in this application may be partially or fully implemented by a special purpose computer created by configuring a general-purpose computer to execute one or more particular functions embodied in computer programs. The functional blocks and flowchart elements described above serve as software specifications, which may be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

The example embodiment or each example embodiment should not be understood as a limiting/restrictive of inventive concepts. Rather, numerous variations and modifications are possible in the context of the present disclosure, in particular those variants and combinations which may be inferred by the person skilled in the art with regard to achieving the object for example by combination or modification of individual features or elements or method steps that are described in connection with the general or specific part of the description and/or the drawings, and, by way of combinable features, lead to a new subject matter or to new method steps or sequences of method steps, including insofar as they concern production, testing and operating methods. Further, elements and/or features of different example embodiments may be combined with each other and/or substituted for each other within the scope of this disclosure.

We claim:

1. A method for managing emergency response services, the method being performed at a system, the method comprising:
receiving emergency information related to a patient, the emergency information comprising one or more of:
description of injuries of the patient,
description of health issues of the patient, and
visual media depicting present state of the patient;
determining a location of the patient;
assessing health condition of the patient based at least in part on the received emergency information;
selecting a category of hospitals and a category of ambulances, based at least in part on the assessed health condition of the patient; and
sending a first alert to one or more ambulances of the selected category of ambulances, the first alert comprising the location of the patient, and one or more hospitals from the selected category of hospitals.

2. The method of claim 1, further comprising:
receiving a profile of the patient, wherein the profile comprises one or more of:
(i) a physical profile of the patient, (ii) a gender profile of the patient, and (iii) medical history of the patient,
wherein the physical profile comprises one or more of physique and age of the patient, the gender profile comprises gender of the patient, and the medical history of the patient comprises past health information of the patient;
receiving disaster-related information for the location, wherein the disaster-related information comprises information on one or more of:
(i) epidemics in the location, (ii) hazards in the location, and (iii) weather and environmental conditions in the location;
wherein the health condition of the patient is assessed based at least in part on the emergency information, the profile of the patient, and the disaster related information.

3. The method of claim 2, wherein the selection of the category of hospitals further comprising:
determining medical interventions required for the patient based at least in part on the assessed health condition of the patient and the profile of the patient,
the medical interventions comprising: surgery, treatment, therapy, or a combination thereof;
determining the time within which the determined medical interventions need to be provided to the patient;
determining equipment required for the determined medical interventions;
determining healthcare professionals required for the determined medical interventions and the determined equipment; and
identifying the category of hospitals from a plurality of categories of hospitals, based on the determined medical interventions, the determined time, the determined equipment, and the determined healthcare professionals.

4. The method of claim 3, wherein the selection of the category of ambulances further comprising:
determining on-board medical facilities required for the patient in a desired ambulance, based at least in part, on the determined time and the determined medical interventions;
determining on-board equipment required in the desired ambulance based at least in part on the determined medical facilities and the physical profile of the patient;
determining on-board healthcare professionals required for the determined medical facilities and the determined equipment;
identifying the category of ambulances from a plurality of categories of ambulances, based on the determined on-board medical facilities, the determined on-board equipment, and the determined on-board healthcare professionals.

5. The method of claim 1, wherein the one or more ambulances of the selected category of ambulances are determined based at least in part on factors comprising:
locations of ambulances of the selected category of ambulances;
available routes from the respective locations of the ambulances to the location of the patient;
estimated weather conditions on the available routes;
estimated wait times at traffic signals on the available routes;
estimated traffic conditions on the available routes;
estimated travel times from the respective locations of the ambulances to the location of the patient;
estimated VIP movement on the available routes; and
estimated public events in the vicinity of the available routes.

6. The method of claim 1, wherein the one or more hospitals are determined based at least in part on factors comprising:
locations of hospitals of the selected category of hospitals;
available routes from the location of the patient to the respective locations of the hospitals;
estimated weather conditions on the available routes;
estimated wait times at traffic signals on the available routes;
estimated traffic conditions on the available routes;
estimated travel times from the location of the patient to the respective locations of the hospitals;
estimated VIP movement on the available routes; and
estimated public events in the vicinity of the available routes.

7. The method of claim 4, wherein a first category of hospitals is selected when the assessed health condition is a first health condition, and a second category of hospitals is selected when the assessed health condition is a second health condition; and
wherein a first category of ambulances is selected when the assessed health condition is the first health condition and the profile of the patient is a first profile, and a second category of ambulances is selected when the assessed health condition is the first health condition and the profile of the patient is a second profile.

8. The method of claim 2, wherein the system stores a corresponding record for each subscriber of emergency response services, wherein a record comprises:
unique identifier of a corresponding subscriber; and
profile of the corresponding subscriber,
wherein the profile of the corresponding subscriber comprises one or more of:
physical profile of the corresponding subscriber;
gender profile of the corresponding subscriber; and
medical history of the corresponding subscriber.

9. The method of claim 8, wherein the patient is a subscriber, and wherein the method further comprises receiving, from a user device, a unique identifier corresponding to the patient, wherein the unique identifier is one of an alphanumeric code, a numeric code and a QR code;
prompting a user of the user device to confirm that the subscriber is in an emergency situation at the location from which the unique identifier is sent;
receiving a confirmation from the user device;
determining a location of the user device upon receiving the confirmation; and
prompting the user to send emergency information related to the subscriber;
wherein the emergency information related to the patient is received in response to the prompting of the user to send the emergency information related to the subscriber,
wherein the determining of the location of the patient comprises determining the location of user device as the location of patient, and
wherein the receiving of the profile of the patient includes retrieving the profile of the patient from corresponding record of the patient using the unique identifier corresponding to the patient.

10. The method of claim 9, wherein the record corresponding to the patient also comprises:
one or more emergency contacts of the patient; and
at least one of (i) medical insurance details of the patient, wherein the medical insurance details comprise details of one or more medical insurance accounts linked to the patient, and (ii) details of one or more digital wallets of the patient, wherein the one or more digital wallets contain funds for emergency services; and
wherein the method further comprises:
sending a second alert to a hospital, the second alert comprising the present health condition of the patient;
sending a communication to the one or more emergency contacts of the patient, the communication comprising one or more of:
real-time location of the subscriber;
real-time location of the ambulance;
location of the hospital;
contact details of the ambulance; and
contact details of the hospital;
guiding the ambulance to the hospital;
facilitating automated registration of the patient with the hospital using the unique identifier of the patient and one of (i) a medical insurance account of the patient, and (ii) a digital wallet of the patient.

11. The method of claim 2, wherein the health condition of the patient is assessed using a first multimodal machine-learning model,
wherein inputs of the first multimodal machine-learning model include the emergency information related to the patient, the profile of the patient, and the disaster related information for the location,
wherein the inputted emergency information comprises:
at least one of (a) the description of the injuries of the patient, and (b) the description of the health issues of the patient; and
the visual media depicting present state of the patient;
wherein the inputted profile of the patient input comprises:
the physical profile of the patient, the gender profile of the patient, and the medical history of the patient;
wherein the inputted disaster related information for the location comprises:
epidemics in the location, hazards in the location, and weather and environmental conditions in the location;
wherein one or more outputs of the multimodal machine-learning model include the assessed health condition of the patient.

12. A system for managing emergency response services, the system comprising:
a memory to store instructions;
one or more processors to execute the instructions stored in the memory to cause the system to perform the actions of:
receiving emergency information related to a patient, the emergency information comprising one or more of:
description of injuries of the patient,
description of health issues of the patient, and
visual media depicting present state of the patient;
determining a location of the patient;
assessing health condition of the patient based at least in part on the received emergency information;
selecting a category of hospitals and a category of ambulances, based at least in part on the assessed health condition of the patient; and
sending a first alert to one or more ambulances of the selected category of ambulances, the first alert comprising the location of the patient, and one or more hospitals from the selected category of hospitals.

13. The system of claim 12, the system to further perform the actions of:
receiving a profile of the patient, wherein the profile comprises one or more of:
(i) a physical profile of the patient, (ii) a gender profile of the patient, and (iii) medical history of the patient,
wherein the physical profile comprises one or more of physique and age of the patient, the gender profile comprises gender of the patient, and the medical history of the patient comprises past health information of the patient;
receiving disaster-related information for the location, wherein the disaster-related information comprises information on one or more of:
(i) epidemics in the location, (ii) hazards in the location, and (iii) weather and environmental conditions in the location;
wherein the health condition of the patient is assessed based at least in part on the emergency information, the profile of the patient, and the disaster related information.

14. The system of claim 13, wherein the selection of the category of hospitals further comprising:
determining medical interventions required for the patient based at least in part on the assessed health condition of the patient and the profile of the patient,
the medical interventions comprising: surgery, treatment, therapy, or a combination thereof;
determining the time within which the determined medical interventions need to be provided to the patient;
determining equipment required for the determined medical interventions;
determining healthcare professionals required for the determined medical interventions and the determined equipment; and
identifying the category of hospitals from a plurality of categories of hospitals, based on the determined medical interventions, the determined time, the determined equipment, and the determined healthcare professionals.

15. The system of claim 14, wherein the selection of the category of ambulances further comprising:
determining on-board medical facilities required for the patient in a desired ambulance, based at least in part, on the determined time and the determined medical interventions;
determining on-board equipment required in the desired ambulance based at least in part on the determined medical facilities and the physical profile of the patient;
determining on-board healthcare professionals required for the determined medical facilities and the determined equipment;
identifying the category of ambulances from a plurality of categories of ambulances, based on the determined on-board medical facilities, the determined on-board equipment, and the determined on-board healthcare professionals.

16. The system of claim 12, wherein the one or more ambulances of the selected category of ambulances are determined based at least in part on factors comprising:
locations of ambulances of the selected category of ambulances;
available routes from the respective locations of the ambulances to the location of the patient;
estimated weather conditions on the available routes;
estimated wait times at traffic signals on the available routes;
estimated traffic conditions on the available routes;
estimated travel times from the respective locations of the ambulances to the location of the patient;
estimated VIP movement on the available routes; and
estimated public events in the vicinity of the available routes.

17. The system of claim 12, wherein the one or more hospitals are determined based at least in part on factors comprising:
locations of hospitals of the selected category of hospitals;
available routes from the location of the patient to the respective locations of the hospitals;
estimated weather conditions on the available routes;
estimated wait times at traffic signals on the available routes;
estimated traffic conditions on the available routes;
estimated travel times from the location of the patient to the respective locations of the hospitals;
estimated VIP movement on the available routes; and
estimated public events in the vicinity of the available routes.

18. The system of claim 13, wherein the health condition of the patient is assessed using a first multimodal machine-learning model,
wherein inputs of the first multimodal machine-learning model include the emergency information related to the patient, the profile of the patient, and the disaster related information for the location,
wherein the inputted emergency information comprises:
at least one of (a) the description of the injuries of the patient, and (b) the description of the health issues of the patient; and
the visual media depicting present state of the patient;
wherein the inputted profile of the patient input comprises:
the physical profile of the patient, the gender profile of the patient, and the medical history of the patient;
wherein the inputted disaster related information for the location comprises:
epidemics in the location, hazards in the location, and weather and environmental conditions in the location;
wherein one or more outputs of the multimodal machine-learning model include the assessed health condition of the patient.

19. The system of claim 15, wherein a first category of hospitals is selected when the assessed health condition is a first health condition, and a second category of hospitals is selected when the assessed health condition is a second health condition; and
wherein a first category of ambulances is selected when the assessed health condition is the first health condition and the profile of the patient is a first profile, and a second category of ambulances is selected when the assessed health condition is the first health condition and the profile of the patient is a second profile.

20. The system of claim 13, wherein the system stores a corresponding record for each subscriber of emergency response services, wherein a record comprises:
unique identifier of a corresponding subscriber; and
profile of the corresponding subscriber,
wherein the profile of the corresponding subscriber comprises one or more of:
physical profile of the corresponding subscriber;
gender profile of the corresponding subscriber; and
medical history of the corresponding subscriber
wherein the patient is a subscriber, and the system to further perform the actions of:
receiving, from a user device, a unique identifier corresponding to the patient, wherein the unique identifier is one of an alphanumeric code, a numeric code and a QR code;
prompting a user of the user device to confirm that the subscriber is in an emergency situation at the location from which the unique identifier is sent;
receiving a confirmation from the user device;
determining a location of the user device upon receiving the confirmation; and
prompting the user to send emergency information related to the subscriber;
wherein the emergency information related to the patient is received in response to the prompting of the user to send the emergency information related to the subscriber,
wherein the determining of the location of the patient comprises determining the location of user device as the location of patient, and
wherein the receiving of the profile of the patient includes retrieving the profile of the patient from corresponding record of the patient using the unique identifier corresponding to the patient; and
wherein the record corresponding to the patient also comprises:
one or more emergency contacts of the patient; and
at least one of (i) medical insurance details of the patient, wherein the medical insurance details comprise details of one or more medical insurance accounts linked to the patient, and (ii) details of one or more digital wallets of the patient, wherein the one or more digital wallets contain funds for emergency services; and
wherein the system to further perform the actions of:
sending a second alert to a hospital, the second alert comprising the present health condition of the patient;
sending a communication to the one or more emergency contacts of the patient, the communication comprising one or more of:
real-time location of the subscriber;
real-time location of the ambulance;
location of the hospital;
contact details of the ambulance; and
contact details of the hospital;
guiding the ambulance to the hospital;
facilitating automated registration of the patient with the hospital using the unique identifier of the patient and one of (i) a medical insurance account of the patient, and (ii) a digital wallet of the patient.
